(19) United States
(12) Patent Application Publication
(10) Pub. No.: US 2026/0265205 A1
(43) Pub. Date: Sep. 10, 2026

(54) FLUORINATED MU-OPIOID RECEPTOR AGONISTS

(71) Applicant: The United State of America, as represented by the Secretary, Department of Health and Human, Bethesda, MD (US)

(72) Inventors: Michael Michaelides, Fallston, MD (US); Juan L. Gomez, Towson, MD (US); Kenner C. Rice, Bethesda, MD (US); Agnieszka Sulima, Poolesville, MD (US); Michael H. Baumann, Baltimore, MD (US)

(73) Assignee: The United State of America, as represented by the Secretary, Department of Health and Human, Bethesda, MD (US)

(21) Appl. No.: 19/165,933

(22) PCT Filed: Dec. 4, 2023
(86) PCT No.: PCT/US2023/082355

§ 371 Date: Sep. 16, 2025

Related U.S. Application Data
(60) Provisional application No. 63/452,879, filed on Mar. 17, 2023.

(51) Int. Cl. C07D 235/12; A61K 31/4184; A61K 51/04; A61P 25/04; A61P 25/36; C07B 59/00; C07D 235/08; C07D 235/10

(52) U.S. Cl. CPC C07D 235/12; A61K 31/4184; A61K 51/0453; A61P 25/04; A61P 25/36; C07B 59/002; C07D 235/08; C07D 235/10

(57) ABSTRACT

The invention provides a fluorinated compound of Formula (I): a deuterated or tritiated variant thereof, or a pharmaceutically acceptable salt thereof, wherein at least one C—H bond on the compound of Formula (I) is replaced with a C—F bond or a C—¹⁸F bond. The invention further provides compositions comprising the fluorinated compound, a deuterated or tritiated variant thereof, or a pharmaceutically acceptable salt thereof, and methods of using the fluorinated compound, a deuterated or tritiated variant thereof, or a pharmaceutically acceptable salt thereof.

### CROSS-REFERENCE TO RELATED APPLICATION

This patent application claims the benefit of U.S. Provisional Patent Application No. 63/452,879, filed Mar. 17, 2023, which is incorporated by reference in its entirety herein.

### FEDERALLY SPONSORED RESEARCH OR DEVELOPMENT

This invention was made with Government support under project number ZIA000069 by the National Institutes of Health, National Institute on Drug Abuse. The Government has certain rights in the invention.

### BACKGROUND OF THE INVENTION

Opiates have long been used and studied for their analgesic properties, and synthetic mu opioid receptor (MOR) agonist medications are the most effective analgesics available. However, such medications have known adverse effects including, but not limited to, constipation, respiratory depression, hyperalgesia, allodynia, and abuse liability. As such, there is a need to develop novel MOR agonists with lower adverse effect profiles. A critical tool for such an endeavor involves the development of a MOR-selective radiotracer for in vivo target engagement studies using positron emission tomography (PET).

The only MOR selective PET agonist radiotracer developed to date is [¹¹C]carfentanil. [¹¹C]carfentanil has been used in many studies to measure MOR binding in humans and laboratory animals. Despite its use, [¹¹C]carfentanil has two key limitations: (i) [¹¹C]carfentanil has very high potency, which necessitates achieving high specific activity in its radiosynthesis, and (ii) use of [¹¹C]carfentanil is restricted to PET studies in centers with on-site cyclotrons. Accordingly, an alternative MOR selective agonist PET radiotracer, which has longer half-life and lower potency than [¹¹C]carfentanil, is desirable.

Etonitazene is a specific and potent (approximately 1,000 times the potency of morphine) MOR agonist. Etonitazene and other nitazene analogues vary greatly in their potency and efficacy for MOR agonism. The etonitazene analogues, reported to date, have not been studied extensively and are classified as Schedule I narcotic controlled substances. Etonitazene and some of its analogues are believed to have strong dependency potential and have been shown to have a tendency to produce profound respiratory depression. For this reason, etonitazene and its analogues are not currently used in humans.

Thus, there remains a need to discover new alternatives to carfentanil and etonitazene, which can be used as potential therapeutics in the treatment of pain and/or as PET radiotracers for target engagement studies. The invention provides such compounds. These and other advantages of the invention, as well as additional inventive features, will be apparent from the description of the invention provided herein.

### BRIEF SUMMARY OF THE INVENTION

The invention provides a fluorinated compound of Formula (I):

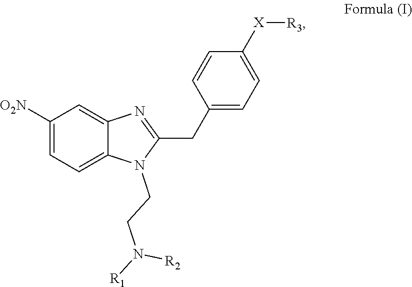

a deuterated or tritiated variant thereof, or a pharmaceutically acceptable salt thereof, wherein X is optionally present and, when present, is —O—, —S—, or —NR₄—, wherein each of R₁, R₂, R₃, and R₄ is independently selected from hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, and a combination thereof, and wherein at least one C—H bond on the compound of Formula (I) is replaced with a C—F bond or a C—¹⁸F bond.

The invention also provides a pharmaceutical composition comprising a fluorinated compound of Formula (I), a deuterated or tritiated variant thereof, or a pharmaceutically acceptable salt thereof, and a pharmaceutically acceptable carrier.

The invention further provides a method of treating or preventing pain in a subject, the method comprising administering to the subject a fluorinated compound of Formula (I), a deuterated or tritiated variant thereof, or a pharmaceutically acceptable salt thereof, or a pharmaceutical composition comprising a fluorinated compound of Formula (I), a deuterated or tritiated variant thereof, or a pharmaceutically acceptable salt thereof.

The invention also provides a method of treating opioid addiction or opioid use disorder in a subject, the method comprising administering to the subject a fluorinated compound of Formula (I), a deuterated or tritiated variant thereof, or a pharmaceutically acceptable salt thereof, or a pharmaceutical composition comprising a fluorinated compound of Formula (I), a deuterated or tritiated variant thereof, or a pharmaceutically acceptable salt thereof.

The invention further provides a method of monitoring mu-opioid receptor binding in a subject, the method comprising administering to the subject a fluorinated compound of Formula (I), a deuterated or tritiated variant thereof, or a pharmaceutically acceptable salt thereof, or a pharmaceutical composition comprising a fluorinated compound of Formula (I), a deuterated or tritiated variant thereof, or a pharmaceutically acceptable salt thereof, wherein at least one C—H bond on the fluorinated compound is replaced with a C—¹⁸F bond, and using positron emission tomography (PET) to determine mu-opioid receptor binding in the subject.

The invention also provides a method of anesthetizing a subject, the method comprising administering to the subject a fluorinated compound of Formula (I), a deuterated or tritiated variant thereof, or a pharmaceutically acceptable salt thereof, or a pharmaceutical composition comprising a fluorinated compound of Formula (I), a deuterated or tritiated variant thereof, or a pharmaceutically acceptable salt thereof.

### BRIEF DESCRIPTION OF THE SEVERAL VIEWS OF THE DRAWINGS

FIG. 1 provides a heat map, as measured by % inhibition of control specific binding or activity, for a pharmacological target screen of fluornitrazene (FNZ).

FIG. 2 provides a graph showing the results of a competitive mu-opioid receptor binding study in rat membranes.

FIGS. 3A-3D provide graphs showing the effect of FNZ on MOR signaling (FIG. 3A), KOR signaling (FIG. 3C), and DOR signaling (FIG. 3D), as determined by cAMP inhibition % of morphine, as well as the effect of FNZ on β-arrestin recruitment (MOR) (FIG. 3B)).

FIGS. 4A-4C provide fluorescent imaging of brain tissue treated with a [³⁵S]GTPγS cocktail with the indicated drugs (DAMGO 10 μM or FNZ 10 μM), without drugs (basal condition). FIG. 4A shows the tissue treated without drugs (basal condition), FIG. 4B shows the tissue treated with DAMGO (10 μM), and FIG. 4C shows the tissue treated with FNZ (10 μM).

FIGS. 5A and 5B provide graphs showing the effect of FNZ on the MOR G protein activity in the brain, as measured in the caudate putamen (CPu) (FIG. 5A) and the nucleus accumbens (NAc) (FIG. 5B).

FIG. 6 shows the occupancy of MORs in the brain and concentration of FNZ in the blood plasma and brain tissue, as exhibited by rats treated with a vehicle control (VEH), FNZ (1 μg/kg, 10 μg/kg, and 100 μg/kg), and naloxone (NAL).

FIG. 7 provides a graph showing FNZ metabolism in mouse liver microsomes fortified with NADPH and in a negative control without NADPH.

FIGS. 8A-8D provide the metabolite profile of FNZ metabolism in mouse liver microsomes fortified with NADPH. The metabolite profile was assessed with high resolution mass spectrometry (FIG. 8A), where the major proposed metabolite is set forth in FIG. 8B and the minor proposed metabolites are set forth in FIGS. 8C and 8D.

FIGS. 9A and 9B provide graphs showing the results of an efflux transporter panel exhibited by FNZ (100 nM) (FIG. 9A) and FNZ (10 μM) (FIG. 9B), as measured by % inhibition.

FIGS. 10A and 10B show the lateral view (FIG. 10A) and dorsal view (FIG. 10B) of the accumulation of [¹⁸F]FNZ in the brain over 60 minutes of a rat treated with [¹⁸F]FNZ.

FIGS. 11A and 11B provide graphs showing the time activity curves of [¹⁸F]FNZ accumulation in the caudate putamen (CPu) (MOR-rich region) and cerebellum (CB) (MOR-poor region). FIG. 11A shows the CPu uptake and CB uptake of [¹⁸F]FNZ in rats, which had been pretreated with vehicle (SAL) or naltrexone (NLX). FIG. 11B shows the CPu/CB ratio of [¹⁸F]FNZ uptake in rats, which had been pretreated with vehicle (SAL) or naltrexone (NLX).

FIG. 12. shows the accumulation of [¹⁸F]FNZ specific binding (i.e., displaceable by naloxone) in the MOR-rich brain regions.

FIG. 13 shows multiple views of the [³H]FNZ (1 μg/kg) brain distribution at approximately seven minutes post injection in a rat.

FIG. 14 shows multiple view of the [³H]FNZ (10 μg/kg) brain distribution at approximately 30 minutes post injection.

FIGS. 15A and 15B provide graphs showing the locomotor activity in mice, as exhibited by mice treated with FNZ and Morphine. FIG. 15A shows the cumulative distance traveled over each 60 minute session at each dose. FIG. 15B shows the time-course of distance traveled during each 60 minute session.

FIG. 16 provides a graph showing the pulse oximetry results, as measured by arterial O₂, for rats treated with FNZ over a time period of 120 minutes post subcutaneous injection. The vertical line denotes FNZ injection time.

FIGS. 17A and 17B show the results for the β-arrestin2 recruitment assay and the mini-Gai recruitment assay, respectively, described in Example 14. Data in the graphs are shown as mean receptor activation±standard error of the mean (SEM) from three independent experiments (n=3).

FIGS. 18A and 18B show the results for the β-arrestin2 recruitment assay and the mini-Gai recruitment assay in HEK293 cells, respectively, described in Example 14.

FIGS. 19A-19C provide graphs showing the results of analgesia, catalepsy, and

hypothermia in rats, which have been treated with FNZ, as described in Example 15. FIG. 19A provides the hot plate latency as measured by % maximum possible effect (% MPE).

FIG. 19B provides the catalepsy score as measured by % maximum possible effect (% MPE).

FIG. 19C provides the change in body temperature of the rats.

FIGS. 20A and 20B provide the results of the von Frey 30 minute post-injection analgesia test and the von Frey 4-6 hour post-injection hyperalgesia test, as described in Example 16.

FIGS. 21A-21F provide the results of the heroin self-administration and food self-administration tests, described in Example 17. The heroin self-administration results are set forth in FIG. 21A (30 minutes), 21C (60 minutes), and 21E (180 minutes), and the food self-administration results are set forth in 21B (30 minutes), 21D (60 minutes), and 21F (180 minutes).

FIGS. 22A and 22B provide the FNZ-induced dose-dependent oxygen responses (FIG. 22A) and fentanyl-induced dose-dependent oxygen responses (FIG. 22B) in the nucleus accumbens (NAc) space and the subcutaneous (SC) space, as described in Example 18. Mean (±SEM) changes in oxygen levels with filled symbols showing values significantly different from pre-injection baseline (p<0.01). n=numbers of averaged responses.

FIGS. 23A and 23B provide comparative results of the FNZ-induced dose-dependent oxygen response and the fentanyl-induced dose-dependent oxygen responses in the nucleus accumbens (NAc) space (FIG. 23A) and the subcutaneous (SC) space (FIG. 23B), as described in Example 18. Mean (±SEM) changes in oxygen levels with filled symbols showing values significantly different from pre-injection baseline (p<0.01). n=numbers of averaged responses.

### DETAILED DESCRIPTION OF THE INVENTION

The invention provides a fluorinated compound of Formula (I):

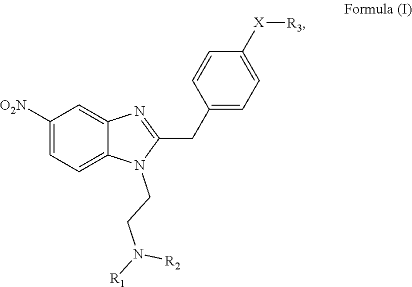

a deuterated or tritiated variant thereof, or a pharmaceutically acceptable salt thereof,
wherein X is optionally present and, when present, is —O—, —S—, or —NR₄—,
wherein each of R₁, R₂, R₃, and R₄ is independently selected from hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, and a combination thereof, and
wherein at least one C—H bond on the compound of Formula (I) is replaced with a C—F bond or a C—¹⁸F bond.

In some embodiments, at least one C—H bond on the compound of Formula (I) that is replaced with a C—F bond or a C—¹⁸F bond is replaced with a C—F bond. Alternatively, or additionally, at least one C—H bond on the compound of Formula (I) that is replaced with a C—F bond or a C—¹⁸F bond is replaced with a C—¹⁸F bond.

Thus, at least one C—H bond on the compound of Formula (I) is replaced with a C—F bond or a C—¹⁸F bond. In other words, the compound of Formula (I) has at least one substituent that is fluorine (F) or 18flourine (¹⁸F). In some embodiments, exactly one C—H bond on the compound of Formula (I) is replaced with a C—F bond or a C—¹⁸F bond. In other embodiments, more than one (e.g., 2, 3, 4, 5, or more) C—H bond on the compound of Formula (I) is replaced with a C—F bond or a C—¹⁸F bond.

For example, any one of the three sp3 hybridized carbons depicted in the compound of Formula (I) can have an F or ¹⁸F substituent, any one of the seven sp2 hybridized carbons depicted in the compound of Formula (I) can have an F or ¹⁸F substituent, any one of R₁, R₂, R₃, and R₄ can have an F or ¹⁸F substituent, or any combination thereof, so long as at least one C—H bond on the compound of Formula (I) is replaced with a C—F bond or a C—¹⁸F bond. Thus, in some embodiments, at least one C—H bond on the compound of Formula (I) that is replaced with a C—F bond or a C—¹⁸F bond is sp3 hybridized or sp2 hybridized. Alternatively, or additionally, at least one C—H bond on the compound of Formula (I) that is replaced with a C—F bond or a C—¹⁸F bond is sp3 hybridized.

In some embodiments, the compound of Formula (I) is deuterated or tritiated. In other words, any hydrogen atom of the compound of Formula (I) can be replaced with a deuterium atom or a tritium atom. In some embodiments, the compound of Formula (I) is not deuterated or tritiated.

In some embodiments, at least one C—H bond of substituent R₁, R₂, R₃, and/or R₄ on the compound of Formula (I) is replaced with a C—F bond or a C—¹⁸F bond. In certain embodiments, at least one C—H bond of substituent R₁ on the compound of Formula (I) is replaced with a C—F bond or a C—¹⁸F bond.

In the fluorinated compound of Formula (I), X is optionally present and, when present, is —O—, —S—, or —NR₄—. In some embodiments, X is not present in the compound of Formula (I). In other words, X can be a bond from the phenyl ring to R₃. Thus, in embodiments where X is not present, the fluorinated compound of Formula (I) is of formula:

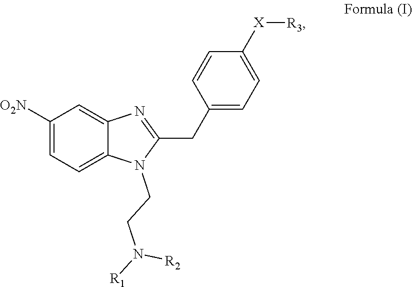

a deuterated or tritiated variant thereof, or a pharmaceutically acceptable salt thereof,
wherein each of R₁, R₂, and R₃ is independently selected from hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, and a combination thereof, and wherein at least one C—H bond on the compound of Formula (I) is replaced with a C—F bond or a C—¹⁸F bond.

In other embodiments, X is present in the compound of Formula (I). In embodiments where X is present, the fluorinated compound of Formula (I) is of formula:

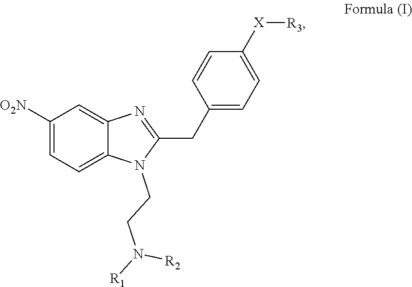

a deuterated or tritiated variant thereof, or a pharmaceutically acceptable salt thereof,
wherein X is —O—, —S—, or —NR₄—, wherein each of R₁, R₂, R₃, and R₄ is independently selected from hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, and a combination thereof, and wherein at least one C—H bond on the compound of Formula (I) is replaced with a C—F bond or a C—¹⁸F bond. In certain embodiments, X is —O—.

Each of R₁, R₂, R₃, and R₄ is independently selected from hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, and a combination thereof. In some embodiments, each of R₁, R₂, and R₃ is independently selected from a C₁-6 alkyl group, a C₂-6 alkenyl group, a C₂-6 alkynyl group, a C₁-6 heteroalkyl group, a C₃-6 cycloalkyl group, a C₃-6 heterocycloalkyl group, an aryl group, a heteroaryl group, an alkaryl group, an arylalkyl group, and a combination thereof. In certain embodiments, each of R₁, R₂, and R₃ is independently selected from C₁-C₆ alkyl, C₂-C₆ alkenyl, and C₂-C₆ alkynyl. For example, each of R₁, R₂, and R₃ can be independently selected from methyl, ethyl, and propyl.

Substituent R₁ of the fluorinated compound of Formula (I) can be hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a combination thereof. In some embodiments, R₁ is a C₁₋₆ alkyl group, a C₂₋₆ alkenyl group, a C₂₋₆ alkynyl group, a C₁-6 heteroalkyl group, a C₃₋₆ cycloalkyl group, a C₃₋₆ heterocycloalkyl group, an aryl group, a heteroaryl group, an alkaryl group, an arylalkyl group, and a combination thereof. In certain embodiments, R₁ is C₁-C₆ alkyl, C₂-C₆ alkenyl, or C₂-C₆ alkynyl. For example, R₁ can be methyl, ethyl, or propyl, and preferably ethyl. Any R₁ substituent described herein can be substituted with F or ¹⁸F such that at least one C—H bond on the compound of Formula (I) is replaced with a C—F bond or a C—¹⁸F bond. In some embodiments, R₁ and R₂ are the same.

Substituent R₂ of the fluorinated compound of Formula (I) can be hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a combination thereof. In some embodiments, R₂ is a C₁₋₆ alkyl group, a C₂₋₆ alkenyl group, a C₂₋₆ alkynyl group, a C₁-6 heteroalkyl group, a C₃₋₆ cycloalkyl group, a C₃₋₆ heterocycloalkyl group, an aryl group, a heteroaryl group, an alkaryl group, an arylalkyl group, and a combination thereof. In certain embodiments, R₂ is C₁-C₆ alkyl, C₂-C₆ alkenyl, or C₂-C₆ alkynyl. For example, R₂ can be methyl, ethyl, or propyl, and preferably ethyl. Any R₂ substituent described herein can be substituted with F or ¹⁸F such that at least one C—H bond on the compound of Formula (I) is replaced with a C—F bond or a C—¹⁸F bond.

Substituent R₃ of the fluorinated compound of Formula (I) can be hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a combination thereof. In some embodiments, R₃ is a C₁₋₆ alkyl group, a C₂₋₆ alkenyl group, a C₂₋₆ alkynyl group, a C₁-6 heteroalkyl group, a C₃₋₆ cycloalkyl group, a C₃₋₆ heterocycloalkyl group, an aryl group, a heteroaryl group, an alkaryl group, an arylalkyl group, and a combination thereof. In certain embodiments, R₃ is C₁-C₆ alkyl, C₂-C₆ alkenyl, or C₂-C₆ alkynyl. For example, R₃ can be methyl, ethyl, or propyl, and preferably ethyl. Any R₃ substituent described herein can be substituted with F or ¹⁸F such that at least one C—H bond on the compound of Formula (I) is replaced with a C—F bond or a C—¹⁸F bond. In some embodiments, R₃ is substituted with F or ¹⁸F such that at least one C—H bond on the compound of Formula (I) is replaced with a C—F bond or a C—¹⁸F bond. In some embodiments, R₃ is the same as R₁ and/or R₂.

Substituent R₄ of the fluorinated compound of Formula (I), when present, can be hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a combination thereof. In some embodiments, R₄ is a C₁₋₆ alkyl group, a C₂₋₆ alkenyl group, a C₂₋₆ alkynyl group, a C₁-6 heteroalkyl group, a C₃₋₆ cycloalkyl group, a C₃₋₆ heterocycloalkyl group, an aryl group, a heteroaryl group, an alkaryl group, an arylalkyl group, and a combination thereof. In certain embodiments, R₄ is C₁-C₆ alkyl, C₂-C₆ alkenyl, or C₂-C₆ alkynyl. For example, R₄ can be methyl, ethyl, or propyl, and preferably ethyl. Any R₄ substituent described herein can be substituted with F or ¹⁸F such that at least one C—H bond on the compound of Formula (I) is replaced with a C—F bond or a C—¹⁸F bond. In some embodiments, R₄ is the same as R₁ and/or R₂.

In some embodiments, the fluorinated compound is of Formula (Ia):

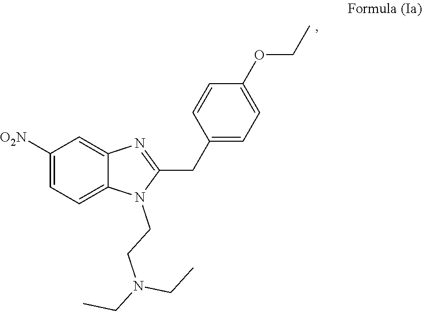 [SMILES: CCOc1ccc(Cc2nc3cc([N+](=O)[O-])ccc3n2CCN(CC)CC)cc1]

a deuterated or tritiated variant thereof, or a pharmaceutically acceptable salt thereof,
wherein at least one C—H bond on the compound of Formula (Ia) is replaced with a C—F bond or a C—¹⁸F bond. In some embodiments, at least one C—H bond on the compound of Formula (Ia) that is replaced with a C—F bond or a C—¹⁸F bond is replaced with a C—F bond. Alternatively, or additionally, at least one C—H bond on the compound of Formula (Ia) that is replaced with a C—F bond or a C—¹⁸F bond is replaced with a C—¹⁸F bond.

In some embodiments, the fluorinated compound is of Formula (Ib):

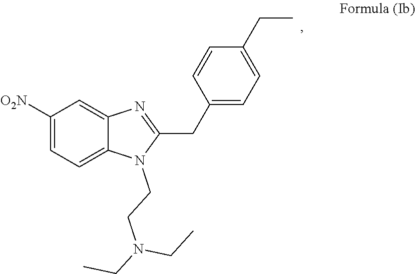 [SMILES: CCc1ccc(Cc2nc3cc([N+](=O)[O-])ccc3n2CCN(CC)CC)cc1]

a deuterated or tritiated variant thereof, or a pharmaceutically acceptable salt thereof,
wherein at least one C—H bond on the compound of Formula (Ib) is replaced with a C—F bond or a C—¹⁸F bond. In some embodiments, at least one C—H bond on the compound of Formula (Ib) that is replaced with a C—F bond or a C—¹⁸F bond is replaced with a C—F bond. Alternatively, or additionally, at least one C—H bond on the compound of Formula (Ib) that is replaced with a C—F bond or a C—¹⁸F bond is replaced with a C—¹⁸F bond.

For example, the fluorinated compound can be:

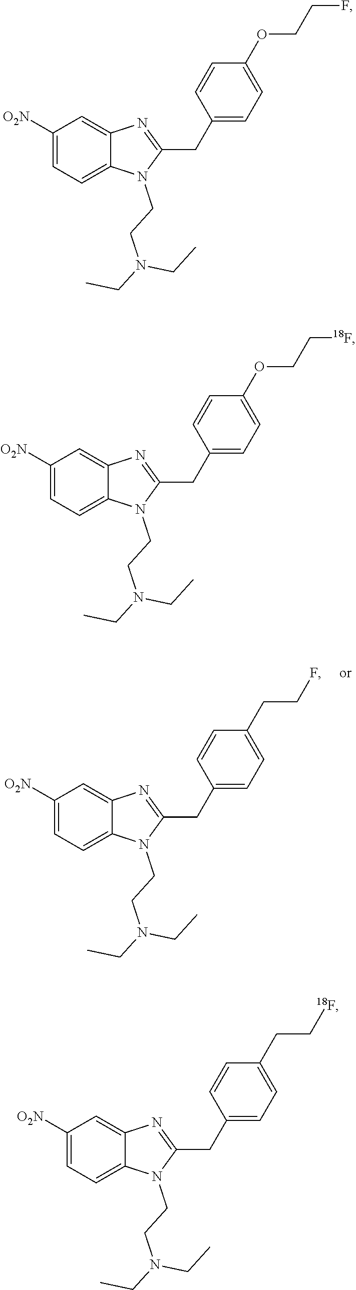

a deuterated or tritiated variant thereof, or a pharmaceutically acceptable salt thereof.

As used herein, the term “alkyl group” refers to a saturated hydrocarbon group, having the specified number of carbon atoms, usually from 1 to about 6 carbon atoms, e.g., about 1, about 2, about 3, or about 4 carbon atoms. Examples of alkyl groups include methyl, ethyl, propyl (e.g., isopropyl or n-propyl), butyl (e.g., isobutyl, n-butyl, tert-butyl, or sec-butyl), pentyl, or hexyl.

As used herein, the term “alkenyl group” refers to an alkyl group described herein comprising at least one unsaturated double bond, and having the specified number of carbon atoms, usually from 2 to about 6 carbon atoms, e.g., about 2, about 3, or about 4 carbon atoms.

As used herein, the term “alkynyl group” refers to an alkyl group described herein comprising at least one unsaturated triple bond, and having the specified number of carbon atoms, usually from 2 to about 6 carbon atoms, e.g., about 2, about 3, or about 4 carbon atoms.

As used herein, the term “heteroalkyl group” refers to an alkyl group described herein further containing from 1 to about 3 heteroatoms chosen from N, O, and S, with remaining substituent atoms being carbon, usually from about 1 to about 6 carbon atoms, e.g., about 1, about 2, about 3, or about 4 carbon atoms. Examples of heteroalkyl groups are alkoxy groups, ethereal groups, thioalkyl groups, or thioethereal groups.

As used herein, the term “cycloalkyl group” refers to a saturated hydrocarbon ring group, having the specified number of carbon atoms, usually from 3 to about 6 carbon atoms, e.g., about 3, about 4, about 5, or about 7 carbon atoms. Examples of cycloalkyl groups include cyclopropyl, cyclobutyl, cyclopentyl, or cyclohexyl.

As used herein, the term “heterocyclyl group” refers to a cycloalkyl group, described herein, containing from 1 to about 3 heteroatoms chosen from N, O, and S, with remaining ring atoms being carbon. Examples of heterocyclyl groups include piperazine groups, furan groups, thiazole groups and the like.

As used herein, the term “aryl group” refers to a stable monocyclic or polycyclic, substituted or unsubstituted aromatic ring having 5 to 60 ring carbon atoms, e.g., phenyl, tolyl, xylyl, naphthyl, phenanthryl, and anthracenyl.

As used herein, the term “heteroaryl group” refers to a stable monocyclic aromatic ring having the indicated number of ring atoms which contains from 1 to 3, or in some aspects, from 1 to 2, heteroatoms chosen from N, O, and S, with remaining ring atoms being carbon, or a stable bicyclic or tricyclic system containing at least one 5- to 7-membered aromatic ring which contains from 1 to 3, or in some aspects, from 1 to 2, heteroatoms chosen from N, O, and S, with remaining ring atoms being carbon. Monocyclic heteroaryl groups typically have from 5 to 7 ring atoms, in some aspects, bicyclic heteroaryl groups are 9- to 10-membered heteroaryl groups, that is, groups containing 9 or 10 ring atoms in which one 5- to 7-member aromatic ring is fused to a second aromatic or non-aromatic ring. Exemplary heteroaryl groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, oxazolyl, benzofuranyl, benzothiazolyl, benzolhiophenyl, benzoxadiazolyl, dihydrobenzodioxynyl, furanyl, imidazolyl, indolyl, isothiazolyl, and isoxazolyl.

As used herein, the term “alkaryl group” refers to an aryl group or heteroaryl group being bound to the structure via an alkyl group or heteroalkyl group.

As used herein, the term “arylalkyl group” refers to an alkyl group or heteroalkyl group being bound to the structure via an aryl group or heteroaryl group.

As used herein, the term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts.

The invention also provides a composition, e.g., a pharmaceutical composition, comprising a fluorinated compound of Formula (I) (e.g., the compound of Formula (I), (Ia), or (Ib)), a deuterated or tritiated variant thereof, or a pharmaceutically acceptable salt thereof, as described herein, and optionally a carrier therefor, e.g., a pharmaceutically acceptable carrier such as, for example, water or saline.

In some embodiments, the composition, e.g., the pharmaceutical composition, further comprises one or more pharmaceutically acceptable excipients. For example, the fluorinated compound of Formula (I) (e.g., the compound of Formula (I), (Ia), or (Ib)), a deuterated or tritiated variant thereof, or a pharmaceutically acceptable salt thereof, as described herein can be formulated for parenteral administration, such as IV administration or administration into a body cavity or lumen of an organ. Compositions for injection will commonly comprise a solution of the fluorinated compound of Formula (I) (e.g., the compound of Formula (I), (Ia), or (Ib)), a deuterated or tritiated variant thereof, or a pharmaceutically acceptable salt thereof, as described herein, dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition, e.g., the pharmaceutical composition, can contain any suitable concentration of the fluorinated compound of Formula (I) (e.g., the compound of Formula (I), (Ia), or (Ib)), a deuterated or tritiated variant thereof, or a pharmaceutically acceptable salt thereof, as described herein. The concentration of the fluorinated compound of Formula (I) (e.g., the compound of Formula (I), (Ia), or (Ib)), a deuterated or tritiated variant thereof, or a pharmaceutically acceptable salt thereof, as described herein in the composition can vary widely, and will be selected primarily based on fluid volumes, viscosities, body weight, and the like, in accordance with the particular mode of administration selected and the patient's needs. For example, the concentration of the fluorinated compound of Formula (I) (e.g., the compound of Formula (I), (Ia), or (Ib)), a deuterated or tritiated variant thereof, or a pharmaceutically acceptable salt thereof, as described herein in the composition, e.g., the pharmaceutical composition, can be selected for administering 0.01 ng/kg to 1 mg/kg of the fluorinated compound to the subject, administering 0.1 ng/kg to 100 μg/kg of the fluorinated compound to the subject, administering 1 ng/kg to 100 μg/kg of the fluorinated compound to the subject, or administering 10 ng/kg to 10 μg/kg of the fluorinated compound to the subject.

In some embodiments, the composition, e.g., the pharmaceutical composition, can be used as a therapeutic (e.g., in the treatment of pain). In such embodiments, the composition, e.g., the pharmaceutical composition, can comprise any suitable amount of the fluorinated compound of Formula (I) (e.g., a compound of Formulae (I), (Ia), or (Ib)), as described herein. For example, the composition, e.g., the pharmaceutical composition, can comprise about 0.1 μg to about 1 g, for example, about 1 μg to about 1 g, about 1 mg to about 500 mg, about 0.1 μg to about 100 mg, about 1 μg to about 100 mg, about 1 mg to about 100 mg, about 1 mg to about 50 mg, about 1 mg to about 1 g, about 10 mg to about 500 mg, or about 50 mg to about 500 mg of the fluorinated compound of Formula (I) (e.g., the compound of Formula (I), (Ia), or (Ib)), as described herein.

In some embodiments, the composition, e.g., the pharmaceutical composition, can be used as a tracer (e.g., in the monitoring of a mu-opioid receptor). In such embodiments, the composition, e.g., the pharmaceutical composition, can comprise any suitable amount of the fluorinated compound of Formula (I) (e.g., the compound of Formula (I), (Ia), or (Ib)), as described herein. For example, the composition, e.g., the pharmaceutical composition, can comprise about 0.1 ng to about 1 mg, for example, about 1 ng to about 1 mg, about 1 ng to about 500 μg, about 0.1 ng to about 100 μg, about 1 ng to about 100 μg, about 1 ng to about 50 μg, about 10 ng to about 500 μg, or about 50 ng to about 500 μg of the fluorinated compound of Formula (I) (e.g., the compound of Formula (I), (Ia), or (Ib)), as described herein.

The fluorinated compound of Formula (I) (e.g., the compound of Formula (I), (Ia), or (Ib)), a deuterated or tritiated variant thereof, or a pharmaceutically acceptable salt thereof, as described herein, or the composition, e.g., the pharmaceutical composition, described herein can have a therapeutic use or a diagnostic use. For example, the fluorinated compound of Formula (I) (e.g., the compound of Formula (I), (Ia), or (Ib)), a deuterated or tritiated variant thereof, or a pharmaceutically acceptable salt thereof, as described herein, or the composition, e.g., the pharmaceutical composition, described herein can be used therapeutically as an analgesic (i.e., an agent to treat, prevent, or relieve pain), an antinocioceptive agent (i.e., an agent to treat, prevent, or relieve the body's sensory nervous system response to toxic stimuli such as, for example, harmful chemicals, mechanical injuries, and adverse temperatures), a mu-opioid receptor agonist (i.e., an agent that binds to and modulates a mu-opioid receptor), or an opioid subsitution therapeutic (i.e., an agent for treating opioid addiction or opioid use disorder). Thus, the fluorinated compound of Formula (I) (e.g., the compound of Formula (I), (Ia), or (Ib)), a deuterated or tritiated variant thereof, or a pharmaceutically acceptable salt thereof, as described herein, or the composition, e.g., the pharmaceutical composition, described herein can be used to treat or prevent any disease or condition associated with nociception, stress, temperature, respiration, endocrine activity, gastrointestinal activity, memory, mood, and motivation.

Alternatively, or additionally, the fluorinated compound of Formula (I) (e.g., the compound of Formula (I), (Ia), or (Ib)), a deuterated or tritiated variant thereof, or a pharmaceutically acceptable salt thereof, as described herein, or the composition, e.g., the pharmaceutical composition, described herein can be used diagnostically. For example, the fluorinated compound of Formula (I) (e.g., the compound of Formula (I), (Ia), or (Ib)), a deuterated or tritiated variant thereof, or a pharmaceutically acceptable salt thereof, as described herein, or the composition, can be used as a positron emission tomography (PET) tracer (e.g., to monitor mu-opioid receptor binding in a subject).

In some embodiments, the fluorinated compound of Formula (I) (e.g., the compound of Formula (I), (Ia), or (Ib)), a deuterated or tritiated variant thereof, or a pharmaceutically acceptable salt thereof, as described herein can be used as an alternative to fentanyl or other opioids. Accordingly, in certain embodiments, the fluorinated compound of Formula (I) (e.g., the compound of Formula (I), (Ia), or (Ib)), a deuterated or tritiated variant thereof, or a pharmaceutically acceptable salt thereof, as described herein is used as an analgesic (e.g., a preoperative analgesic), as an anesthesia adjunct (e.g., a regional anesthesia adjunct), for general anesthesia, for postoperative pain control, for acute pain (e.g., moderate to severe acute pain), or a combination thereof. In some embodiments, the fluorinated compound of Formula (I) (e.g., the compound of Formula (I), (Ia), or (Ib)), a deuterated or tritiated variant thereof, or a pharmaceutically acceptable salt thereof, as described herein is used as an anesthetic.

In some embodiments, the fluorinated compound of Formula (I) (e.g., the compound of Formula (I), (Ia), or (Ib)), a deuterated or tritiated variant thereof, or a pharmaceutically acceptable salt thereof, as described herein produces reduced hyperalgesia or does not produce hyperalgesia as compared to fentanyl or other opioids. As used herein, the term “hyperalgesia” refers to a state of nociceptive sensitization, which is characterized by a paradoxical response whereby a patient receiving opioids for the treatment of pain could actually become more sensitive to certain painful stimuli. See, for example, Guichard et al. [Clin. J. Pain, 38(1): 49-57 (2022)]and Lee et al. [Pain Physician, 14:145-161 (2011)]for further discussion of opioid-induced hyperalgesia. In that respect, the fluorinated compound of Formula (I) (e.g., the compound of Formula (I), (Ia), or (Ib)), a deuterated or tritiated variant thereof, or a pharmaceutically acceptable salt thereof, as described herein produces less hyperalgesia than fentanyl or other opioids. In certain embodiments, the fluorinated compound of Formula (I) (e.g., the compound of Formula (I), (Ia), or (Ib)), a deuterated or tritiated variant thereof, or a pharmaceutically acceptable salt thereof, as described herein does not produce hyperalgesia.

In some embodiments, the fluorinated compound of Formula (I) (e.g., the compound of Formula (I), (Ia), or (Ib)), a deuterated or tritiated variant thereof, or a pharmaceutically acceptable salt thereof, as described herein produces reduced allodynia or does not produce allodynia as compared to fentanyl or other opioids. As used herein, the term “allodynia” refers to a condition where a person experiences pain from a stimulus, which doesn't normally cause pain. In certain embodiments, the fluorinated compound of Formula (I) (e.g., the compound of Formula (I), (Ia), or (Ib)), a deuterated or tritiated variant thereof, or a pharmaceutically acceptable salt thereof, as described herein does not produce allodynia.

The invention further provides a method of treating or preventing pain in a subject, the method comprising administering to the subject a fluorinated compound of Formula (I) (e.g., the compound of Formula (I), (Ia), or (Ib)) as described herein, or a composition, e.g., a pharmaceutical composition, described herein. The pain can be associated with any condition or disease. For example, the pain can be associated with a toxic stimuli selected from a harmful chemical, a mechanical injury, an adverse temperature, or a combination thereof. In some embodiments, the fluorinated compound of Formula (I) (e.g., the compound of Formula (I), (Ia), or (Ib)), a deuterated or tritiated variant thereof, or a pharmaceutically acceptable salt thereof, as described herein, or the composition, e.g., the pharmaceutical composition, described herein is administered to treat toxic shock syndrome.

The invention further provides a method of treating opioid addiction or opioid use disorder in a subject, the method comprising administering to the subject a fluorinated compound of Formula (I) (e.g., the compound of Formula (I), (Ia), or (Ib)) as described herein, or a composition, e.g., a pharmaceutical composition, described herein. For example, the fluorinated compound of Formula (I) (e.g., the compound of Formula (I), (Ia), or (Ib)), a deuterated or tritiated variant thereof, or a pharmaceutically acceptable salt thereof, as described herein can be administered as a substitution therapy for opioid addition or opioid use disorder, akin to, e.g., methadone or buprenorphine.

The invention further provides a method of monitoring mu-opioid receptor binding in a subject, the method comprising a fluorinated compound of Formula (I) (e.g., the compound of Formula (I), (Ia), or (Ib)) as described herein, or a composition, e.g., a pharmaceutical composition, described herein, wherein at least one C—H bond on the fluorinated compound is replaced with a C—¹⁸F bond, and using positron emission tomography (PET) to determine mu-opioid receptor binding in the subject. In other words, the fluorinated compound of Formula (I) (e.g., the compound of Formula (I), (la), or (Ib)), a deuterated or tritiated variant thereof, or a pharmaceutically acceptable salt thereof, as described herein, wherein at least one C—H bond on the fluorinated compound is replaced with a C—¹⁸F bond, can be used as a radiotracer for positron emission tomography (PET). As used herein, the phrase “mu-opioid receptor” refers to a class of receptors that modulate (e.g., neuromodulate) physiological functions such as, for example, nociception, stress, temperature, respiration, endocrine activity, gastrointestinal activity, memory, mood, and motivation. In some embodiments, the mu-opioid receptor binding is monitored in the brain of the subject.

The invention also provides a method of anesthetizing a subject, the method comprising administering to the subject a fluorinated compound of Formula (I), a deuterated or tritiated variant thereof, or a pharmaceutically acceptable salt thereof, or a pharmaceutical composition comprising a fluorinated compound of Formula (I), a deuterated or tritiated variant thereof, or a pharmaceutically acceptable salt thereof. For example, the fluorinated compound of Formula (I), a deuterated or tritiated variant thereof, or a pharmaceutically acceptable salt thereof, or a pharmaceutical composition comprising a fluorinated compound of Formula (I), a deuterated or tritiated variant thereof, can be administered to a subject about 3 hours or less (e.g., about 2 hours or less, about 1 hour or less or about 30 minutes or less) before surgery to anesthetize the subject. The fluorinated compound of Formula (I), a deuterated or tritiated variant thereof, or a pharmaceutically acceptable salt thereof can be administered alone or as an adjunct to another anesthetizing agent.

As used herein, the terms “treat,” “treatment,” and “treating” refer to any indicia of success in the treatment or amelioration of a condition (e.g., pain management), including any objective or subjective parameter such as abatement; diminishing of symptoms or making the symptom, injury, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

As used herein, the terms “subject” and “patient” are used interchangeably and refer to any subject for whom diagnosis, treatment, or therapy is desired (e.g., humans). In some embodiments, the subject is a mammal. As used herein, the term “mammal” for purposes of treatment refers to any animal classified as a mammal, including humans, domestic and farm animals, and zoo, sports, or pet animals, such as dogs, horses, cats, cows, sheep, goats, pigs, camels, etc. In some certain embodiments, the mammal is human.

As used herein, the term “administering” refers to parenteral, intravenous, intraperitoneal, intramuscular, intratumoral, intralesional, intranasal (e.g., a nasal spray), or subcutaneous administration, oral administration, administration as a suppository, topical contact (e.g., a transdermal patch), intrathecal administration, or the implantation of a slow-release device, e.g., a mini-osmotic pump, to the subject.

The fluorinated compound of Formula (I) (e.g., the compound of Formula (I), (Ia), or (Ib)), a deuterated or tritiated variant thereof, or a pharmaceutically acceptable salt thereof, as described herein, can be administered to a subject in need thereof in any therapeutically effective amount using any suitable dosing regimen. For example, the methods can include administering the fluorinated compound of Formula (I) (e.g., the compound of Formula (I), (Ia), or (Ib)), a deuterated or tritiated variant thereof, or a pharmaceutically acceptable salt thereof, as described herein, to provide a dose of from about 0.01 ng/kg to about 50 mg/kg to the subject. For example, the fluorinated compound of Formula (I) (e.g., the compound of acceptable salt thereof, as described herein, dose can range from 0.01 ng/kg to 1 mg/kg, from 0.1 ng/kg to 100 μg/kg, from 1 ng/kg to 100 μg/kg, or from 10 ng/kg to 10 μg/kg. The fluorinated compound of Formula (I) (e.g., the compound of Formula (I), (Ia), or (Ib)), a deuterated or tritiated variant thereof, or a pharmaceutically acceptable salt thereof, as described herein, dose can also be outside of these ranges, depending on the application (e.g., severity of the pain being treated or the level of signal needed in a diagnostic test). Frequency of administration can range from multiple doses a day to a single dose per week. In some embodiments, the fluorinated compound of Formula (I) (e.g., the compound of Formula (I), (Ia), or (Ib)), a deuterated or tritiated variant thereof, or a pharmaceutically acceptable salt thereof, as described herein is administered as needed. In some embodiments, the fluorinated compound of Formula (I) (e.g., the compound of Formula (I), (Ia), or (Ib)), a deuterated or tritiated variant thereof, or a pharmaceutically acceptable salt thereof, as described herein, is administered once daily.

As used herein, the phrases “effective amount” and “therapeutically effective amount” refer to a dose of the fluorinated compound of Formula (I) (e.g., the compound of Formula (I), (Ia), or (Ib)), a deuterated or tritiated variant thereof, or a pharmaceutically acceptable salt thereof, as described herein, that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding(1999); Pickar, Dosage Calculations(1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)).

The fluorinated compound of Formula (I) (e.g., the compound of Formula (I), (Ia), or (Ib)), a deuterated or tritiated variant thereof, or a pharmaceutically acceptable salt thereof, as described herein can be administered to the subject using any suitable means including, for example, parenteral, intravenous, intraperitoneal, intramuscular, intratumoral, intralesional, intranasal (e.g., a nasal spray), or subcutaneous administration, oral administration, administration as a suppository, or by topical contact (e.g., a transdermal patch), intrathecal administration, or by the implantation of a slow-release device, e.g., a mini-osmotic pump, to the subject. In some embodiments, the compound of Formula (I) (e.g., the compound of acceptable salt thereof, as described herein is administered (a) by intramuscular injection, (b) by intravenous injection or infusion, (c) transdermally (e.g., by using a transdermal patch), (d) intranasally (e.g., by using a nasal spray), or (e) intrathecally.

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered 1-34 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) In aspect (1) is provided a fluorinated compound of Formula (I):

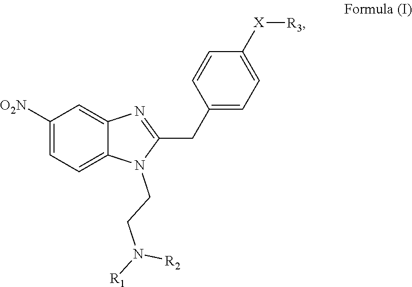

a deuterated or tritiated variant thereof, or a pharmaceutically acceptable salt thereof,
wherein X is optionally present and, when present, is —O—, —S—, or —NR₄—,
wherein each of R₁, R₂, R₃, and R₄ is independently selected from hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, and a combination thereof, and
wherein at least one C—H bond on the compound of Formula (I) is replaced with a C—F bond or a C—¹⁸F bond.

(2) In aspect (2) is provided the fluorinated compound of aspect (1), a deuterated or tritiated variant thereof, or a pharmaceutically acceptable salt thereof, wherein X is —O—.

(3) In aspect (3) is provided the fluorinated compound of aspect (1), a deuterated or tritiated variant thereof, or a pharmaceutically acceptable salt thereof, wherein X is not present.

(4) In aspect (4) is provided the fluorinated compound of any one of aspects (1)-(3), a deuterated or tritiated variant thereof, or a pharmaceutically acceptable salt thereof, wherein each of R₁, R₂, and R₃ is independently selected from C₁-C₆ alkyl, C₂-C₆ alkenyl, and C₂-C₆ alkynyl.

(5) In aspect (5) is provided the fluorinated compound of any one of aspects (1)-(4), a deuterated or tritiated variant thereof, or a pharmaceutically acceptable salt thereof, wherein each of R₁, R₂, and R₃ is independently selected from methyl, ethyl, and propyl.

(6) In aspect (6) is provided the fluorinated compound of any one of aspects (1)-5), a deuterated or tritiated variant thereof, or a pharmaceutically acceptable salt thereof, wherein each of R₁, R₂, and R₃ is ethyl.

(7) In aspect (7) is provided the fluorinated compound of aspect (1), wherein the fluorinated compound is of Formula (Ia):

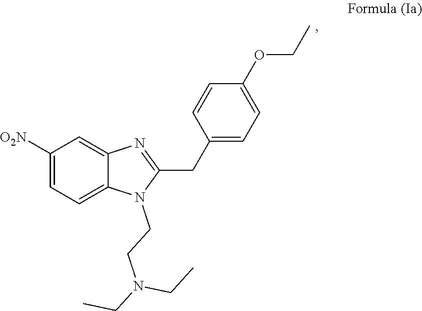 [SMILES: CCOc1ccc(Cc2nc3cc([N+](=O)[O-])ccc3n2CCN(CC)CC)cc1]

a deuterated or tritiated variant thereof, or a pharmaceutically acceptable salt thereof,
wherein at least one C—H bond on the compound of Formula (Ia) is replaced with a C—F bond or a C—¹⁸F bond.

(8) In aspect (8) is provided the fluorinated compound of aspect (1), wherein the fluorinated compound is of Formula (Ib):

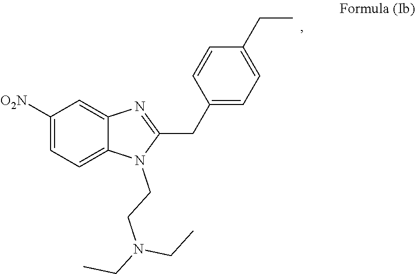 [SMILES: CCc1ccc(Cc2nc3cc([N+](=O)[O-])ccc3n2CCN(CC)CC)cc1]

a deuterated or tritiated variant thereof, or a pharmaceutically acceptable salt thereof,
wherein at least one C—H bond on the compound of Formula (Ib) is replaced with a C—F bond or a C—¹⁸F bond.

(9) In aspect (9) is provided the fluorinated compound of any one of aspects (1)-(8), a deuterated or tritiated variant thereof, or a pharmaceutically acceptable salt thereof, wherein exactly one C—H bond on the compound of Formula (I) is replaced with a C—F bond or a C—¹⁸F bond.

(10) In aspect (10) is provided the fluorinated compound of any one of aspects (1)-(9), a deuterated or tritiated variant thereof, or a pharmaceutically acceptable salt thereof, wherein the at least one C—H bond on the compound of Formula (I) that is replaced with a C—F bond or a C—¹⁸F bond is sp3 hybridized or sp2 hybridized.

(11) In aspect (11) is provided the fluorinated compound of any one of aspects (1)-(10), a deuterated or tritiated variant thereof, or a pharmaceutically acceptable salt thereof, wherein the at least one C—H bond on the compound of Formula (I) that is replaced with a C—F bond or a C—¹⁸F bond is sp3 hybridized.

(12) In aspect (12) is provided the fluorinated compound of any one of aspects (1)-(11), a deuterated or tritiated variant thereof, or a pharmaceutically acceptable salt thereof, wherein the at least one C—H bond on the compound of Formula (I) that is replaced with a C—F bond or a C—¹⁸F bond is replaced with a C—F bond.

(13) In aspect (13) is provided the fluorinated compound of any one of aspects (1)-(11), a deuterated or tritiated variant thereof, or a pharmaceutically acceptable salt thereof, wherein the at least one C—H bond on the compound of Formula (I) that is replaced with a C—F bond or a C—¹⁸F bond is replaced with a C—¹⁸F bond.

(14) In aspect (14) is provided the fluorinated compound of aspect (1), wherein the fluorinated compound is:

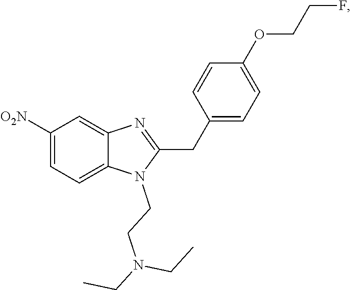 [SMILES: CCN(CC)CCn1c(Cc2ccc(OCCF)cc2)nc2cc([N+](=O)[O-])ccc21]

or a pharmaceutically acceptable salt thereof.

(15) In aspect (15) is provided the fluorinated compound of aspect (1), wherein the fluorinated compound is:

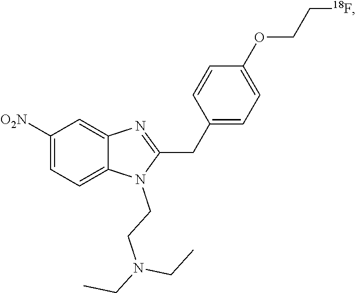

or a pharmaceutically acceptable salt thereof.

(16) In aspect (16) is provided the fluorinated compound of aspect (1), wherein the fluorinated compound is:

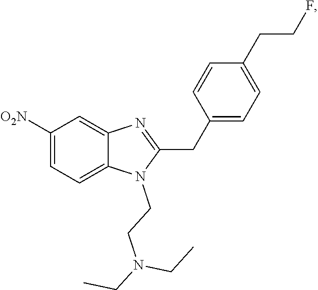 [SMILES: CCN(CC)CCn1c(Cc2ccc(CCF)cc2)nc2cc([N+](=O)[O-])ccc21]

or a pharmaceutically acceptable salt thereof.

(17) In aspect (17) is provided the fluorinated compound of aspect (1), wherein the fluorinated compound is:

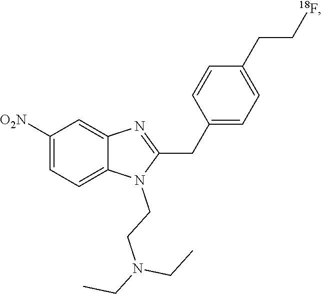

or a pharmaceutically acceptable salt thereof.

(18) In aspect (18) is provided a pharmaceutical composition comprising a fluorinated compound of any one of aspects (1)-(17), a deuterated or tritiated variant thereof, or a pharmaceutically acceptable salt thereof, and a pharmaceutically acceptable carrier.

(19) In aspect (19) is provided the pharmaceutical composition of aspect (18), further comprising one or more pharmaceutically acceptable excipients.

(20) In aspect (20) is provided a fluorinated compound according to any one of aspects (1)-(17), a deuterated or tritiated variant thereof, or a pharmaceutically acceptable salt thereof, or a pharmaceutical composition according to aspect (18) or aspect (19), for use as an analgesic.

(21) In aspect (21) is provided a fluorinated compound according to any one of aspects (1)-(17), a deuterated or tritiated variant thereof, or a pharmaceutically acceptable salt thereof, or a pharmaceutical composition according to aspect (18) or aspect (19), for use as an antinocioceptive agent.

(22) In aspect (22) is provided a fluorinated compound according to any one of aspects (1)-(17), a deuterated or tritiated variant thereof, or a pharmaceutically acceptable salt thereof, or a pharmaceutical composition according to aspect (18) or aspect (19), for use as an anesthetic.

(23) In aspect (23) is provided a fluorinated compound according to any one of aspects (1)-(17), a deuterated or tritiated variant thereof, or a pharmaceutically acceptable salt thereof, or a pharmaceutical composition according to aspect (18) or aspect (19), a deuterated or tritiated variant thereof, for use as a mu-opioid receptor agonist.

(24) In aspect (24) is provided a fluorinated compound according to any one of aspects (1)-(17), a deuterated or tritiated variant thereof, or a pharmaceutically acceptable salt thereof, or a pharmaceutical composition according to aspect (18) or aspect (19), for use in treating opioid addiction or opioid use disorder.

(25) In aspect (25) is provided a fluorinated compound according to any one of aspects (1)-(17), a deuterated or tritiated variant thereof, or a pharmaceutically acceptable salt thereof, or a pharmaceutical composition according to aspect (18) or aspect (19), for use as a positron emission tomography (PET) tracer.

(26) In aspect (26) is provided a method of treating or preventing pain in a subject, the method comprising administering to the subject a fluorinated compound of any one of aspects (1)-17), a deuterated or tritiated variant thereof, or a pharmaceutically acceptable salt thereof, or a pharmaceutical composition according to aspect (18) or aspect (19).

(27) In aspect (27) is provided the method of aspect (26), wherein the pain is associated with a toxic stimuli selected from a harmful chemical, a mechanical injury, an adverse temperature, or a combination thereof.

(28) In aspect (28) is provided a method of treating opioid addiction or opioid use disorder in a subject, the method comprising administering to the subject a fluorinated compound of any one of aspects (1)-(17), a deuterated or tritiated variant thereof, or a pharmaceutically acceptable salt thereof, or a pharmaceutical composition according to aspect (18) or aspect (19).

(29) In aspect (29) is provided a method of monitoring mu-opioid receptor binding in a subject, the method comprising administering to the subject a fluorinated compound of any one of aspects (1)-(17), a deuterated or tritiated variant thereof, or a pharmaceutically acceptable salt thereof, or a pharmaceutical composition according to aspect (18) or aspect (19), wherein at least one C—H bond on the fluorinated compound is replaced with a C—¹⁸F bond, and using positron emission tomography (PET) to determine mu-opioid receptor binding in the subject.

(30) In aspect (30) is provided the method of aspect (29), wherein the mu-opioid receptor binding is monitored in the brain of the subject.

(31) In aspect (31) is provided a method of anesthetizing a subject, the method comprising administering to the subject a fluorinated compound of any one of any one of aspects (1)-(17), a deuterated or tritiated variant thereof, or a pharmaceutically acceptable salt thereof, or a pharmaceutical composition according to aspect (18) or aspect (19).

(32) In aspect (32) is provided the method of any one of aspects (26)-(31), wherein the subject is human.

(33) In aspect (33) is provided the method of any one of aspects (26)-(32), wherein the method comprises administering 0.01 ng/kg to 1 mg/kg of the fluorinated compound, a deuterated or tritiated variant thereof, or a pharmaceutically acceptable salt thereof, to the subject.

(34) In aspect (34) is provided the method of any one of aspects (26)-(32), wherein the method comprises administering 0.1 ng/kg to 100 μg/kg of the fluorinated compound, a deuterated or tritiated variant thereof, or a pharmaceutically acceptable salt thereof, to the subject.

(35) In aspect (35) is provided the method of any one of aspects (26)-(32), wherein the method comprises administering 1 ng/kg to 100 μg/kg of the fluorinated compound, a deuterated or tritiated variant thereof, or a pharmaceutically acceptable salt thereof, to the subject.

(36) In aspect (36) is provided the method of any one of aspects (26)-(32), wherein the method comprises administering 10 ng/kg to 10 μg/kg of the fluorinated compound, a deuterated or tritiated variant thereof, or a pharmaceutically acceptable salt thereof, to the subject.

### EXAMPLES

The following examples further illustrate the invention but, of course, should not be construed as in any way limiting its scope.

### Example 1

This example provides an exemplary synthesis of a compound of Formula (I), namely, fluornitrazene (FNZ), i.e., compound 9, as set forth in Scheme 1.

Melting points were determined on a Thomas-Hoover melting-point apparatus and were uncorrected. Proton and carbon nuclear magnetic resonance (1H and ¹³C NMR) spectra were recorded on a Varian Gemini-400 spectrometer in CDCl₃ (unless otherwise noted) with the values given in ppm (TMS as internal standard) and J (Hz) assignments of 1H resonance coupling. Mass spectra (HRMS) were recorded on a Waters (Milford, MA USA) Xevo-G XS QTof mass spectrometer. Ions were produced using positive ion electrospray (ESI). Thin layer chromatography (TLC) analyses were carried out on Analtech silica gel GHLF 0.25 mm plates using various gradients of CHCl₃/MeOH containing 1% NH₄OH or gradients of EtOAc/n-hexane. Visualization was accomplished under UV light or by staining in an iodine chamber. Flash column chromatography was performed with Fluka silica gel 60 (mesh 220-400). Flash column chromatography was performed using RediSep Rf normal phase silica gel cartridges. Elemental analyses were performed, and the results were within ±0.4% of the theoretical values.

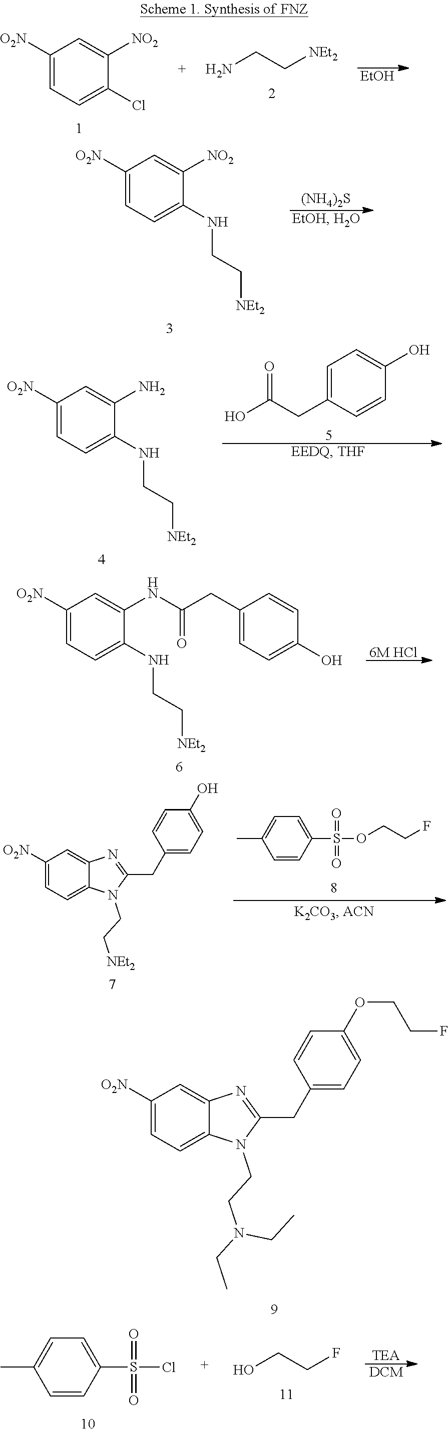

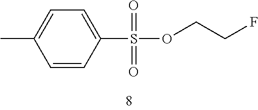

N¹-(2,4-dinitrophenyl)-N2.N²-diethylethane-1,2-diamine (3). To a solution of 1-chloro-2,4-dinitrobenzene (1) (10.13 g, 1 equiv., 50.01 mmol) in ethanol (75 mL) was added N¹,N¹-diethylethane-1,2-diamine (2) (6.974 g, 8.43 mL, 1.2 equiv., 60.01 mmol) under nitrogen and the mixture was stirred at reflux for 20 hours. The reaction mixture was cooled to room temperature, diluted with water (100 mL) and the pH was adjusted to 9-10 with concentrated NH₄OH. The precipitate, a yellow solid, was filtered off, washed with cold water and dried under vacuum in an oven at room temperature to afford 3 (13.8 g). The 1H NMR of the product was in agreement with the literature data [Bucha et al., Asian J. Chem., (2018), 30(1), 29-33].

N¹-(2-(diethylamino)ethyl)-4-nitrobenzene-1,2-diamine (4). To a solution of N¹-(2,4-dinitrophenyl)-N²,N²-diethylethane-1,2-diamine (3) (999 mg, 1 equiv., 3.54 mmol) in ethanol (13 mL) was added dropwise over 30 minutes to a mixture of ammonium bisulfide (1.876 g, 1.88 mL, 45 wt. %, 3.50 equiv., 12.39 mmol), water (14 mL) and ethanol (28 mL) under argon at 65° C. The reaction mixture was heated at 70° C. for 24 hours, cooled to room temperature and the pH was adjusted to 1 with 2N HCl. The solvent was removed under vacuum and the aqueous layer was made basic with concentrated ammonium hydroxide. The aqueous layer was extracted with DCM (4×50 mL), the combined organic layers were dried and concentrated. The resulting residue was purified by flash chromatography using a mixture of CHCl₃/MeOH/NH₄OH as an eluent to yield 4 (0.85 g) as a red oil. The 1H NMR of the product was in agreement with the literature data [Bucha et al., Asian J. Chem., (2018), 30(1), 29-33].

N-(2-((2-(diethylamino)ethyl)amino)-5-nitrophenyl)-2-(4-hydroxyphenyl) acetamide (6). A mixture of N¹-(2-(diethylamino)ethyl)-4-nitrobenzene-1,2-diamine (4) (853 mg, 1 equiv., 3.38 mmol), 2-(4-hydroxyphenyl) acetic acid (5) (566 mg, 1.1 equiv., 3.72 mmol), and EEDQ (836 mg, 1 equiv., 3.38 mmol) in THF (4 mL) was heated at 50° C. for 26 hours under nitrogen. The reaction mixture was cooled to room temperature and the solvent removed under vacuum. The residue, a red oil was treated with hexane (3×10 mL) and vigorously stirred for 10 minutes each time. The hexane washes were discarded and the residue, a red oil, was purified by flash chromatography using a mixture of CHCl₃/MeOH/NH₄OH as an eluent to afford 6 (425 mg) as a red oil. 1H NMR (400 MHZ; CDCl₃): δ 7.93-7.91 (m, 1H), 7.86 (d, J=2.51 Hz, 1H), 7.07 (d, J=8.32 Hz, 2H), 6.69 (d, J=8.32 Hz, 2H), 6.46 (d, J=9.20 Hz, 1H), 5.37 (s, 1H), 3.59 (s, 2H), 3.16-3.14 (m, 2H), 2.66-2.63 (m, 2H), 2.56 (q, J=7.42 Hz, 4H), 1.12 (t, J=7.42 Hz, 6H). ¹³C NMR (101 MHz; CDCl₃): δ 171.2, 156.0, 148.8, 136.6, 130.6, 125.3, 125.0, 123.6, 120.9, 116.4, 109.5, 50.6, 46.2, 42.8, 39.9, 11.2. HRMS ESI (m/z): [M+H]⁺ calcd for C₂₀H₂₇N₄O₄ 387.2030, found 387.2032.

4-((1-(2-(diethylamino)ethyl)-5-nitro-1H-benzo[d]imidazol-2-yl)methyl) phenol (7). N-(2-((2-(diethylamino)ethyl)amino)-5-nitrophenyl)-2-(4-hydroxyphenyl) acetamide (6) (425 mg, 1 equiv., 1.10 mmol) was taken into 18% HCl (7 mL, 0.04 mol) and stirred at 100 C. for 2 hours under nitrogen. The reaction mixture was cooled in an ice-water bath and the pH was adjusted to 8 with concentrated NH₄OH to precipitate the product. A yellow solid was collected, washed with cold water followed by hexane ash and air-dried to give 350 mg of 7, which was additionally purified by crystallization from EtOH to yield 296 mg of light-yellow crystals, mp 186-187° C. 1H NMR (400 MHZ; CDCl₃): δ 8.60 (d, J=2.12 Hz, 1H), 8.18 (dd, J=8.92 and 2.12 Hz, 1H), 7.69 (s, 1H), 7.35 (d, J=8.92 Hz, 1H), 7.00 (d, J=8.43 Hz, 2H), 6.69 (d, J=8.32 Hz, 2H), 4.29 (s, 2H), 4.13 (t, J=6.62 Hz, 2H), 2.60 (t, J=6.62 Hz, 2H), 2.48 (q, J=7.12 Hz, 4H), 0.87 (t, J=7.12 Hz, 6H). ¹³C NMR (101 MHz; CDCl₃): δ 158.1, 155.7, 143.6, 141.4, 139.3, 129.6, 126.3, 118.3, 116.1, 115.8, 109.4, 52.1, 47.5, 43.7, 33.5, 11.7. HRMS ESI (m/z): [M+H]⁺ calcd for C₂₀H₂₅N₄O₃ 369.1927, found 369.1924. Anal. calcd, for C₂₀H₂₄N₄O₃×0.4 H₂O: C 63.95; H 6.65; N 14.91; Found: C 63.83; H 6.44; N 14.87.

2-fluoroethyl 4-methylbenzenesulfonate (8). To a solution of 2-fluoroethan-1-ol (11) (5.0 g, 1 equiv., 78 mmol) in DCM (50 mL) was added tosyl-C₁ (10) (18 g, 1.2 equiv., 94 mmol) followed by triethylamine (11.8 g, 16.3 mL, 1.5 equiv., 117 mmol) at room temperature under nitrogen. The reaction mixture became heterogeneous after 10-15 minutes and the stirring was continued for additional 16 hours. The solvent was removed under vacuum and the residue was taken into ether and water (50 mL). The aqueous layer was additionally extracted with ether (3×30 mL). The combined organic layers were dried, concentrated and the residue was purified by flash chromatography using a mixture of hexane/ethyl acetate to yield 8 (16.2 g) as a colorless oil. 1H NMR (400 MHZ; CDCl₃): δ 7.79 (m, 2H), 7.34 (m, 2H), 4.61 (m, 1H), 4.49 (m, 1H), 4.28 (m, 1H), 4.21 (m, 1H), 2.44 (s, 3H). ¹³C NMR (101 MHz; CDCl₃): δ 145.1, 132.6, 129.9, 127.91, 81.3, 79.6, 68.5, 68.2, 21.6. The 1H NMR of the product was in agreement with the literature data [Bouter et al., Eur. J. Med. Chem., (2022), 232, 1141138].

N,N-diethyl-2-(2-(4-(2-fluoroethoxy)benzyl)-5-nitro-1H-benzo[d]imidazol-1-yl) ethan-1-amine (9). To a mixture of 4-((1-(2-(diethylamino)ethyl)-5-nitro-1H-benzo[d]imidazol-2-yl)methyl) phenol (7) (625 mg, 1 equiv., 1.70 mmol) and potassium carbonate (469 mg, 2 equiv., 3.39 mmol) in acetonitrile (10 mL) was added 2-fluoroethyl 4-methylbenzenesulfonate (8) (555 mg, 1.5 equiv., 2.54 mmol) at room temperature under nitrogen. The reaction mixture was heated at 80° C. for 28 hours. The reaction mixture was cooled to room temperature and the solvent removed under vacuum. The residue was taken into a mixture of water (6 mL) and CHCl₃, the layers were separated and the aqueous layer was additionally extracted with CHCl₃ (3×6 mL). The combined organic layers were dried and concentrated. The light-brown oil was purified by flash chromatography using a mixture of CHCl₃/MeOH/NH₄OH as an eluent to afford 740 mg of 9 (i.e., fluornitrazene (FNZ)) as a yellow oil that solidified upon standing at room temperature. The free base was converted to a hydrochloride salt, colorless needles, 550 mg, mp 183-184° C. 1H NMR (400 MHZ; CDCl₃): δ 60 (d, J=2.09 Hz, 1H), 8.18 (dd, J=8.92 and 2.09 Hz, 1H), 7.33 (d, J=8.90 Hz, 1H), 7.15 (d, J=8.60 Hz, 2H), 6.69 (d, J=8.60 Hz, 2H), 4.78 (t, J=4.15 Hz, 1H), 4.66 (t, J=4.15 Hz, 1H), 4.34 (s, 2H), 4.19 (t, J=4.16 Hz, 1H), 4.12 (t, J=4.16 Hz, 1H), 4.08 (t, J=6.66 Hz, 2H), 2.54 (t, J=6.66 Hz, 2H), 2.45 (q, J=7.12 Hz, 4H), 0.86 (t, J=7.12 Hz, 6H). ¹³C NMR (101 MHz; CDCl₃): δ 157.6, 143.5, 141.9, 139.6, 129.6, 128.1, 118.2, 116.2, 115.1, 109.3, 82.7, 80.9, 67.3, 67.1, 52.1, 47.6, 43.8, 33.7, 11.8. HRMS ESI (m/z): [M+H]⁺ calcd for C₂₂H₂₈N₄O₃F 415.2145, found 415.2148. Anal. calcd, for C₂₂H₂₈CIFN₄O₃ X 0.83EtOH: C 58.09; H 6.8; N 11.45; Found: C 57.97; H 6.67; N 11.47.

### Example 2

This example provides the results of a pharmacological target screen of fluornitrazene (FNZ).

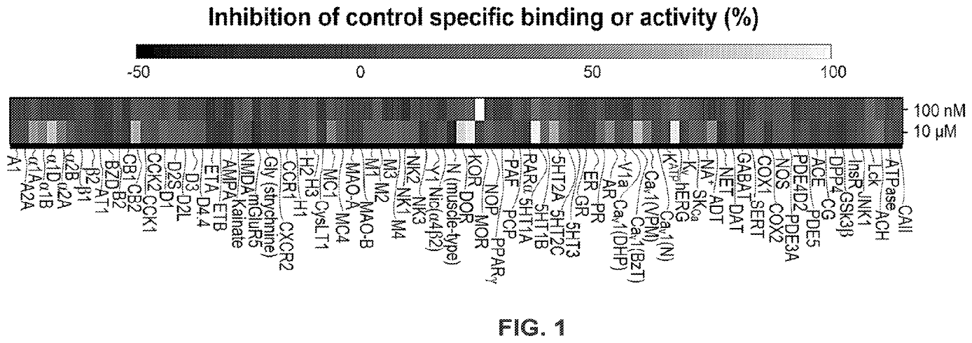

Membrane homogenates from stable cell lines or rat tissues expressing each receptor/enzyme were incubated with the respective radioligand in the absence or presence of FNZ (100 nM or 10 μM). In each experiment, the respective reference compound was tested concurrently with the test compound to assess the assay reliability. Nonspecific binding was determined in the presence of a specific agonist or antagonist at the target. Following incubation, the samples were filtered rapidly under vacuum through glass fiber filters presoaked in buffer and rinsed several times with an ice-cold buffer using a 48-sample or 96-sample cell harvester. The filters were counted for radioactivity in a scintillation counter using a scintillation cocktail, and the results are set forth in FIG. 1.

As is apparent from the results set forth in FIG. 1, FNZ exhibits a selective interaction with the mu-opioid receptor (MOR) at 100 nM.

### Example 3

This example provides a radiolabeled binding experiment of fluornitrazene (FNZ).

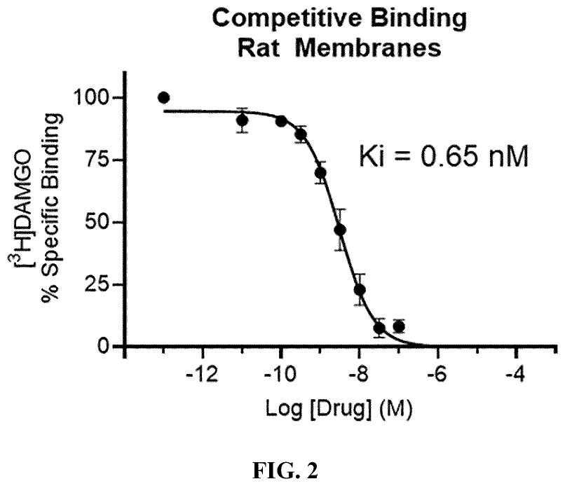

Dissected rat brains (except the cerebellum) were cut and suspended in Tris-HCl 50 mM buffer supplemented with protease inhibitor cocktail (1:1000) and disrupted with a Polytron homogenizer (Kinematica, Basel, Switzerland). Homogenates were centrifuged at 48,000×g (50 minutes, 4° C.) and washed twice in the same conditions to isolate the membrane fraction. Protein was quantified by a bicinchoninic acid method. For competition experiments, membrane suspensions (50 or 100 μg of protein/ml) were incubated with a constant amount of radioligand and the indicated increasing concentrations of FNZ during 2 hours at room temperature. Nonspecific binding was determined in the presence of an exceeding concentration of cold ligand. In all cases, free and membrane-bound radioligand were separated by rapid filtration of 500-μl aliquots in a 96-well plate harvester (PerkinElmer, Boston, MA, USA) and washed with 2 ml of ice-cold Tris-HCl buffer. Microscint-20 scintillation liquid (65 μL/well, PerkinElmer) was added to the filter plates, the filter plates were incubated overnight at room temperature, and radioactivity counts were determined in a MicroBeta2 plate counter (USA) with an efficiency of 41%. One-site competition curves were fitted using Prism 7 (GraphPad Software, La Jolla, CA, USA) and Kᵢ values were calculated using the Cheng-Prusoff equation. For MOR binding, membrane preparations were incubated with 50 mM Tris-HCl (pH 7.4) containing [³H]DAMGO (1 nM, NIDA Drug Supply) and increasing concentrations of FNZ. Non-specific binding was determined in the presence of 100 μM of unlabeled DAMGO, and the competitive binding results are set forth in FIG. 2.

As is apparent from the results set forth in FIG. 2, FNZ binds to mu-opioid receptor (MOR) with high affinity (Kᵢ=0.65 nM).

### Example 4

This example provides the effect on mu-opioid receptor (MOR) signaling, kappa-opioid receptor (KOR) signaling, and delta-opioid receptor (DOR) signaling, as determined by CAMP inhibition % of morphine, as well as the effect on β-arrestin recruitment (MOR), exhibited by cells treated with fluornitrazene (FNZ) in an in vitro assay.

HEK-293T cells (ATCC) cells were maintained in Dulbecco's modified Eagle's medium (DMEM; Sigma-Aldrich, Saint Louis, MO) supplemented with 100 U/ml penicillin (Biowest, Nuaillé, France), 100 mg/ml streptomycin (Biowest), 10% v/v heat-inactivated fetal bovine serum (Invitrogen, Carlsbad, CA, USA), non-essential amino acids (Biowest), 100 mM sodium pyruvate (Biowest), and 2 mM L-glutamine (Biowest), and kept in an incubator at 37° C. and 5% CO₂.

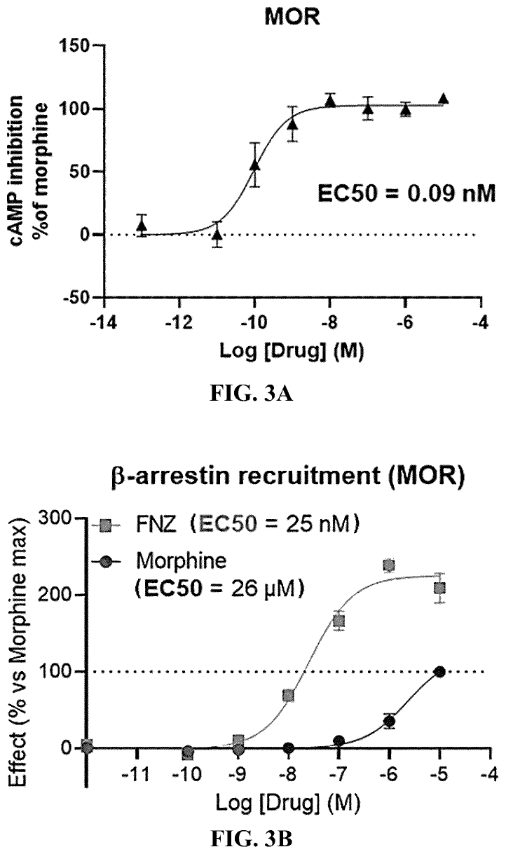

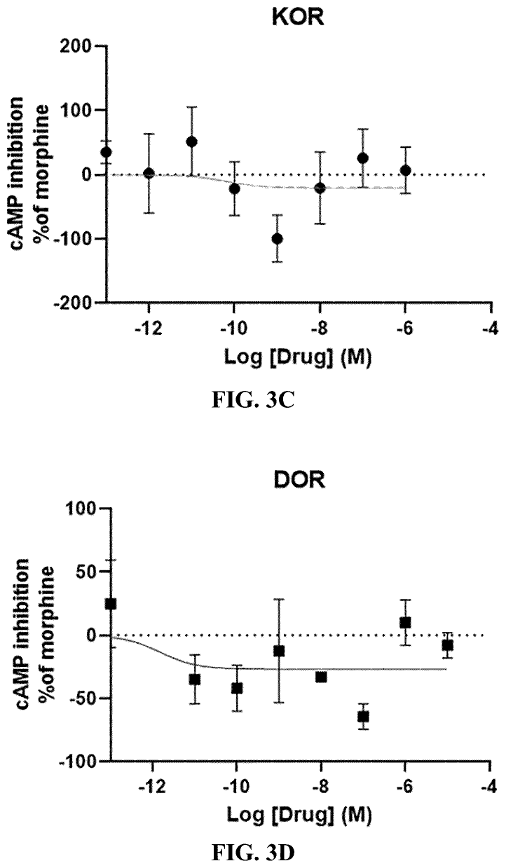

CAMP. Cells were seeded on 100 mm dishes at 2.2× 106 cells/dish 24 hours before transfection and transfected with 10 μg cDNA encoding human MOR, KOR or DOR (cDNA.org) and 10 μg of cDNA encoding for the R-FlincA biosensor using polyethylenimine (PEI; Polysciences Europe GmbH)) in a 1:3 DNA: PEI ratio. Cells were harvested 48 hours after transfection. CAMP accumulation was measured using the R-FlincA (Red Fluorescent indicator for cAMP) biosensor (Ohta et al., Sci Rep., 8(1): 1866 (2018)). On experiment day, cells were harvested, washed in PBS pH 7.4 and re-suspended in PBS containing 5.6 mM glucose and 0.5 U/ml of Adenosine Deaminase (Roche). Cells were then plated in 96-well black flat bottom plates and fluorescence was measured using a CLARIOstar (BMG Labtech) plate reader with excitation at 550 nm and emission at 600-640 nm to establish their baseline fluorescence (FO). Cells were then incubated with increasing amounts of FNZ and forskolin (5 μM) for 10 minutes and their fluorescence was measured again. Relative fluorescence changes were obtained by calculating the fluorescence ratio before and after treatment (F/FO). CAMP inhibition was expressed as % morphine maximum response and concentration-response curves were fitted using Prism 9. The CAMP inhibition results are presented in FIGS. 3A, 3C, and 3D.

β-arrestin recruitment. HEK-293T cells were seeded on 6-well culture plates at 3×10⁶ cells/well and grown in DMEM (Thermo Fisher) supplemented with L-Glutamine 200 mM, Sodium Pyruvate 100 mM and MEM non-Essential Amino Acids 100× (Biowest). 10% fetal bovine serum (FBS; Merck KgaA), streptomycin (100 μg/mL), and penicillin (100 μg/mL) in a controlled environment (37° C., 98% humidity, 5% C₀2). 24 hours after seeding, cells were transfected with split NanoBiT® vectors NB MCS1 (Promega, Madison, WI, United States) fused to β-arrestin2 or hMOR (0.1 μg β-arrestin2-LgBIT cDNA, 2 μg of the hMOR-SmBIT cDNA) using PEI (Polysciences Europe GmbH) in a 1:3 DNA: PEI ratio. 48 hour after transfection, cells were rinsed, harvested, and re-suspended in 4 ml/well of Hanks' Balanced Salt solution (HBSS, Sigma Aldrich). Cells (80 μL/well) were then plated in 96-well white plates (PO-204003, BIOGEN) and immediately treated with increasing concentrations of FNZ, and 5 minutes later, 2 μM coelenterazine (Prolume Ltd) was added and luminescence (490 to 410 nm) was measured during a period of 6 minutes using a CLARIOstar plate reader. β-arrestin recruitment was expressed as % of DAMGO maximum effect and concentration-response curves were fitted using Prism 9. The β-arrestin recruitment results are presented in FIG. 3B.

As is apparent from the results set forth in FIGS. 3A and 3B, FNZ potently inhibits CAMP and activates β-arrestin signaling at the MOR with greater efficacy than morphine. In addition, FNZ does not affect KOR and DOR signaling.

### Example 5

This example provides the agonist stimulated [³⁵S]GTPYS autoradiography of flash-frozen tissue treated with fluornitrazene (FNZ).

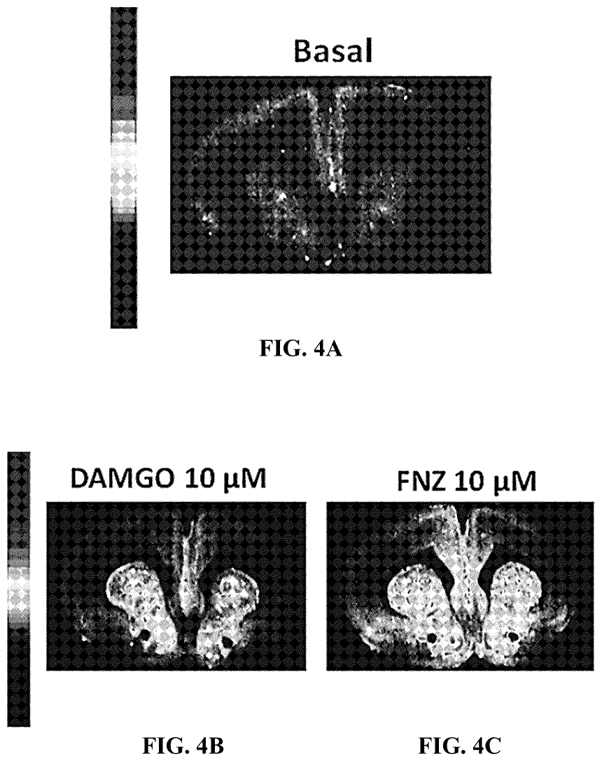

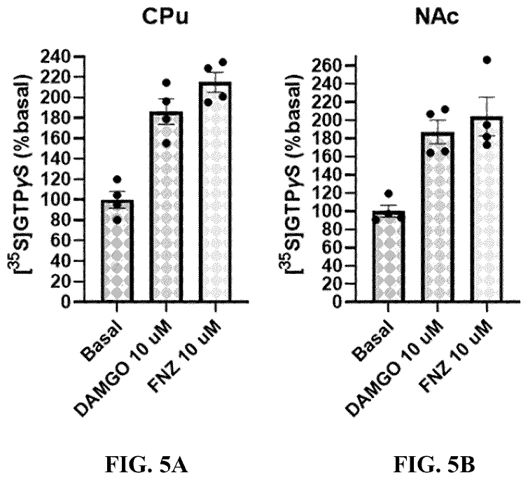

Flash-frozen tissue was sectioned (10 μm) on a cryostat (Leica) and thaw mounted on ethanol cleaned glass slides. Sections were encircled with a hydrophobic membrane using a PAP pen (Sigma-Aldrich). Preincubation buffer (50 mM Tris-HCl, 1 mM EDTA, 5 mM MgCl2, and 100 mM NaCl) was pipetted onto each slide and allowed to incubate for 20 minutes. The preincubation buffer was removed via aspiration and each slide was loaded with guanosine diphosphate (GDP) in the presence of adenosine Al receptor antagonist DPCPX and allowed to incubate for 60 minutes (Preincubation buffer, 2 mM GDP, 1 μM DPCPX, Millipore water). GDP buffer was removed via aspiration and [³⁵S]GTPγS cocktail (GDP buffer, 1.3 mM DTT, 2.7 mM GDP, 1.3 μM DPCPX, 83 PM [³⁵S]GTPYS) with the indicated drugs (DAMGO 10 μM or FNZ 10 μM), without drugs (basal condition), or with a saturated concentration of nonradioactive GTP (for nonspecific binding) was pipetted onto each slide and allowed to incubate for 90 minutes. The [³⁵S]GTPYS cocktail was removed via aspiration and slides were washed in ice-cold washing buffer (50 mM Tris-HCl, 5 mM MgCl2, pH 7.4) for 5 minutes (2×) followed by a 30 second dip in ice-cold deionized water. Hydrophobic membrane was removed with a cotton swab and xylene and slides were placed into a Hypercassette™ covered by a BAS-SR₂₀₄₀ phosphor screen (FujiFilm; GE Healthcare). The slides were exposed to the phosphor screen for 3-5 days and imaged using a phosphor imager (Typhoon FLA 7000; GE Healthcare). The fluorescent imaging results of brain tissue treated without drugs (basal), with DAMGO, and with FNZ are set forth in FIGS. 4A-4C, respectively. In addition, the results are plotted as a function of mu-opioid receptor G-protein activity in the caudate putamen (CPu) (FIG. 5A) and the nucleus accumbens (NAc) (FIG. 5B), as measured by % of basal activity.

As is apparent from the results set forth in FIGS. 4A and 4B and FIGS. 5A and 5B, FNZ activated MOR G-protein activity in the brain with higher efficacy than DAMGO (i.e., a synthetic opioid peptide with high mu-opioid receptor specificity).

### Example 6

This example provides the receptor occupancy and brain/plasma concentrations, exhibited by rats treated with fluornitrazene (FNZ).

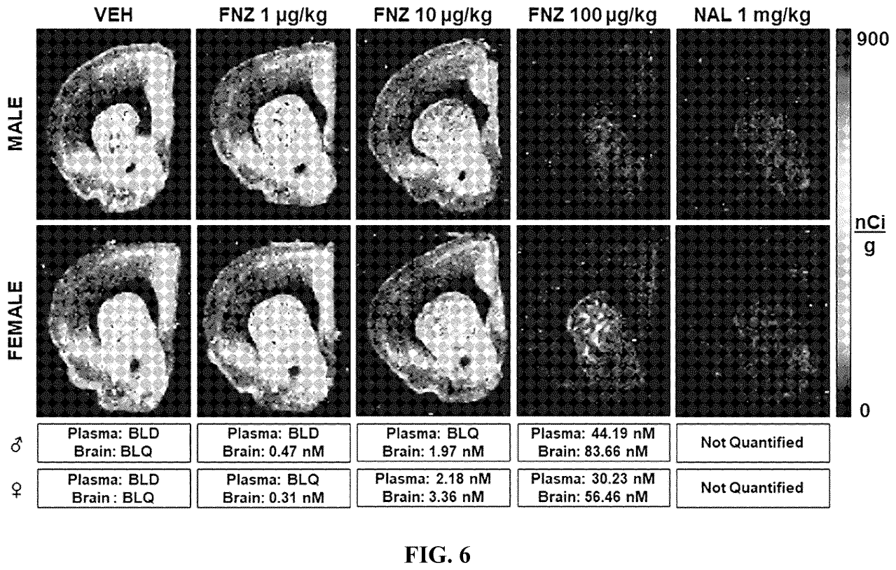

Male and female rats were treated with saline, FNZ (1 μg/kg, 10 μg/kg, or 100 μg/kg administered subcutaneously (SC)), or naloxone (1 mg/kg administered subcutaneously (SC)) 15 minutes before decapitation and tissue extraction. Brains were flash frozen and stored at −80° C. until processed. Flash-frozen brains were separated by hemisphere. One hemisphere used for assessing concentration of FNZ. The other hemisphere was sectioned (20 μm) on a cryostat (Leica) and thaw mounted on ethanol cleaned glass slides. For [³H]DAMGO, 50 mM Tris-HCl buffer containing 5 nM [³H]DAMGO (46 Ci/mmol, NIDA Drug Supply) was pipetted onto slides and allowed to stand for 10 minutes at room temperature. For non-specific binding cold DAMGO (10 μM) was also added. The sections were then washed by 2×30 second washes in their respective Tris buffers. Finally, slides were dipped in ice cold distilled water to remove salts. The slides were exposed to the phosphor screen for 10 days and then imaged using a phosphor imager (Typhoon FLA 7000; GE Healthcare). The digitized images were calibrated using 14C standard slides (American Radiolabeled Chemicals). Regions of interest (ROIs) were hand-drawn based on anatomical landmarks and radioactivity was quantified using Multigauge software (GE Healthcare). The results are set forth in FIG. 6.

As is apparent from the results set forth in FIG. 6, systemic FNZ injections occupy brain mu-opioid receptors (MORs) in vivo. More particularly, MOR occupancy begins to be observed using a 10 μg/kg subcutaneous dose at 15 minutes post injection. This level of MOR in vivo occupancy coincides with low nM levels of FNZ in the brain (i.e., levels at which FNZ shows MOR selectivity).

### Example 7

This example provides the microsomal stability and metabolite identification of fluornitrazene (FNZ).

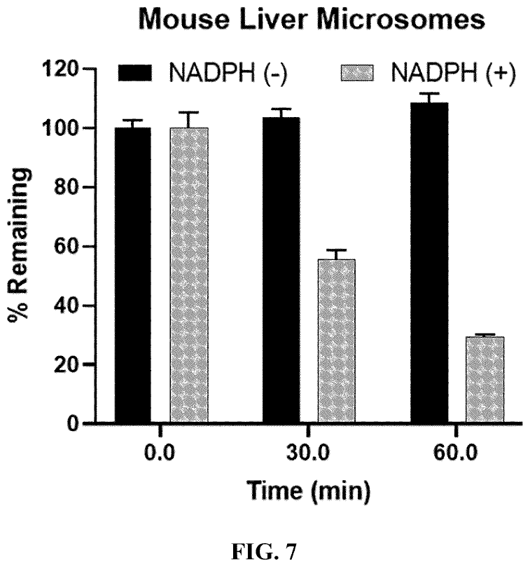

FNZ shows instability to Phase 1 metabolism in mouse liver microsomes fortified with NADPH with 29% remaining at 60 minutes. See FIG. 7. However, FNZ shows complete stability in a negative control without NADPH, suggesting Cytochrome P450 (CYP) dependent metabolism.

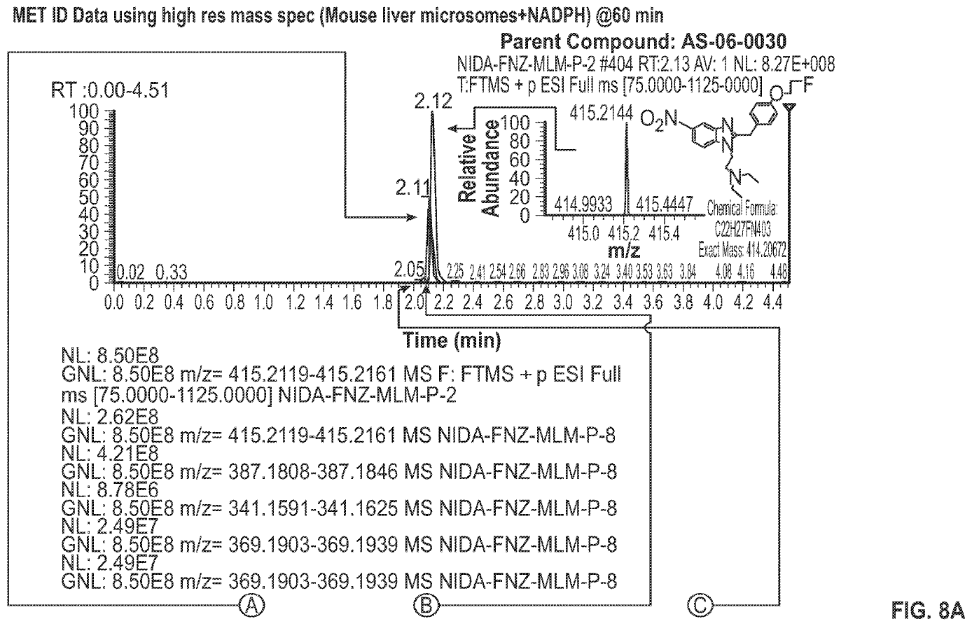

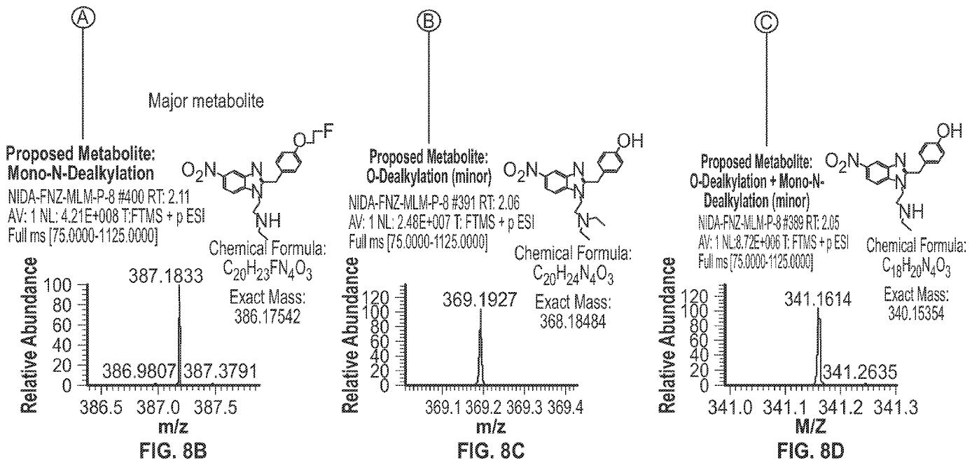

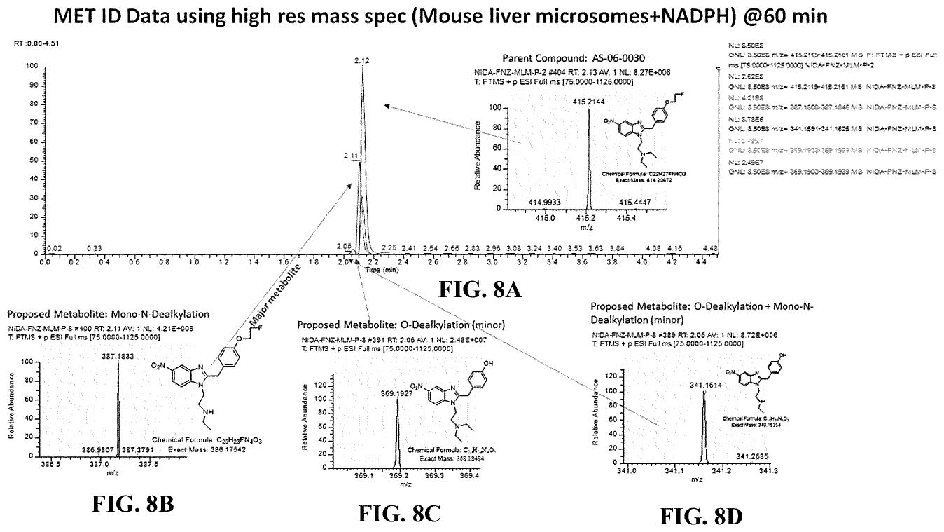

Metabolite identification (MET-ID) was performed on a Dionex ultra high-performance LC system coupled with Q Exactive Focus orbitrap mass spectrometer (Thermo Fisher Scientific Inc., Waltham MA). Separation was achieved using Agilent Eclipse Plus column (100×2.1 mm i.d.; maintained at 35° C.) packed with a 1.8 μM C₁₈ stationary phase. The mobile phase consisted of 0.1% formic acid in water and 0.1% formic acid in acetonitrile. Pumps were operated at a flow rate of 0.3 ml/minutes for 7 minutes using gradient elution. The mass spectrometer controlled by Xcalibur software 4.0.27.13 (Thermo Scientific) was operated with a HESI ion source in positive ionization mode. Metabolites were identified in the full-scan mode (from m/z 50 to 1600) by comparing t=0 samples with t=60 minutes samples and structures were proposed based on the accurate mass information. The resulting metabolite profile of FNZ metabolism in mouse liver microsomes fortified with NADPH is provided in FIG. 8A.

Without wishing to be bound by any particular theory, it is believed that the major proposed metabolite is set forth in FIG. 8B and the minor proposed metabolites are set forth in FIGS. 8C and 8D. The metabolite profile of FNZ suggests that metabolites do not participate actively in its effects in the central nervous system (CNS).

### Example 8

This example provides the results of an efflux transporter panel of fluornitrazene (FNZ).

Membrane homogenates from stable cell lines were pre-incubated with FNZ (100 nM and 10 μM) followed by incubation with appropriate substrate and the presence or absence of an appropriate reference inhibitor. Efflux ratios (E) were calculated based on the apparent B-A and A-B permeability with and without verapamil. In each experiment, the respective reference compound was tested concurrently with the test compound to assess the assay reliability. Fluorescein was used as the cell monolayer integrity marker. Fluorescein permeability assessment (in the A-B direction at pH 7.4 on both sides) was performed after the permeability assay for the test compound. The cell monolayer that had a fluorescein permeability of less than 1.5×10⁻⁶ cms⁻¹ for Caco-2 was considered intact, and the permeability result of the test compound from intact cell monolayer was reported.

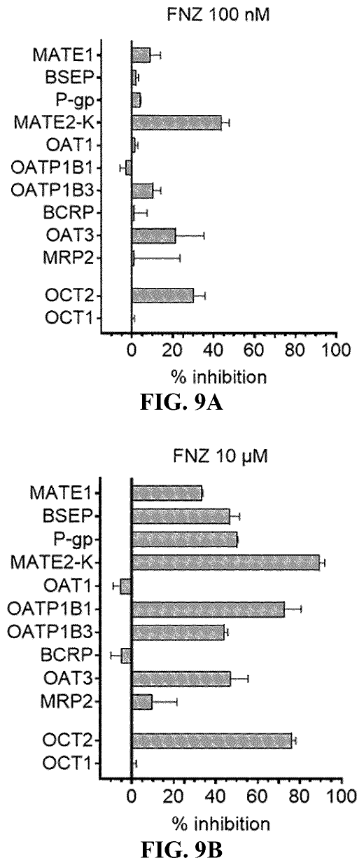

As is apparent from the results set forth in FIGS. 9A and 9B, FNZ is a substrate for efflux transporters with high expression in kidney, which are involved in renal elimination, but is not a substrate for transporters expressed in brain endothelium (e.g., P-gp or BCRP), which are mainly involved in brain uptake.

### Example 9

This example provides an exemplary synthesis of ¹⁸F-fluornitrazene ([¹⁸F]FNZ), as set forth in Scheme 2.

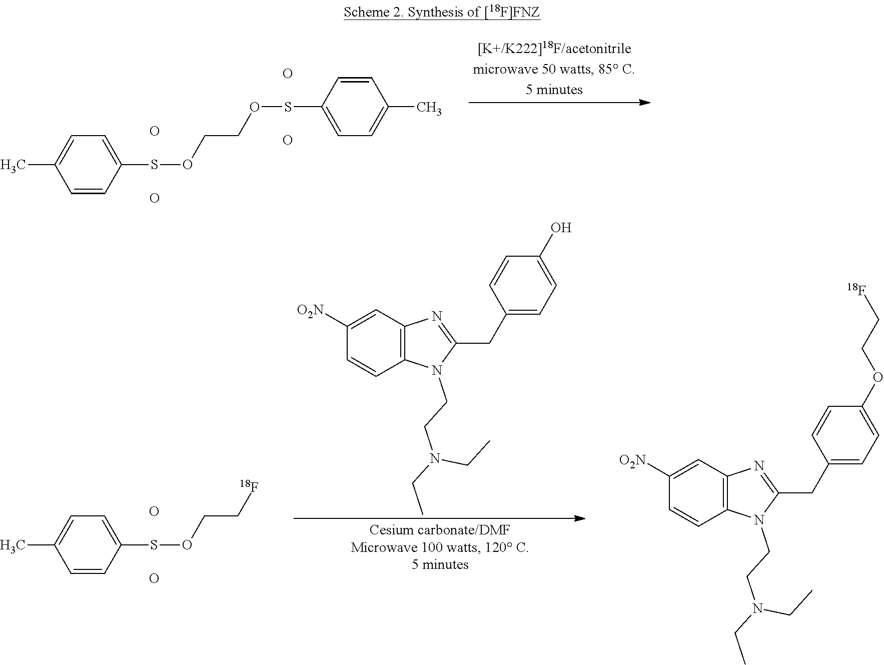

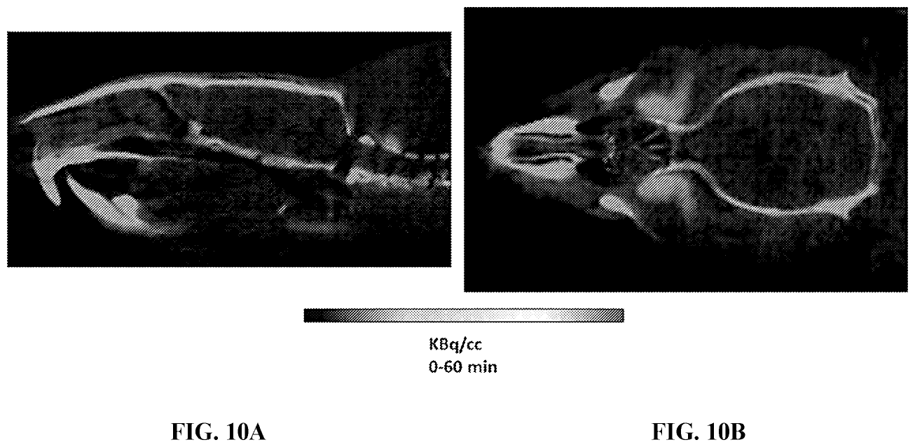

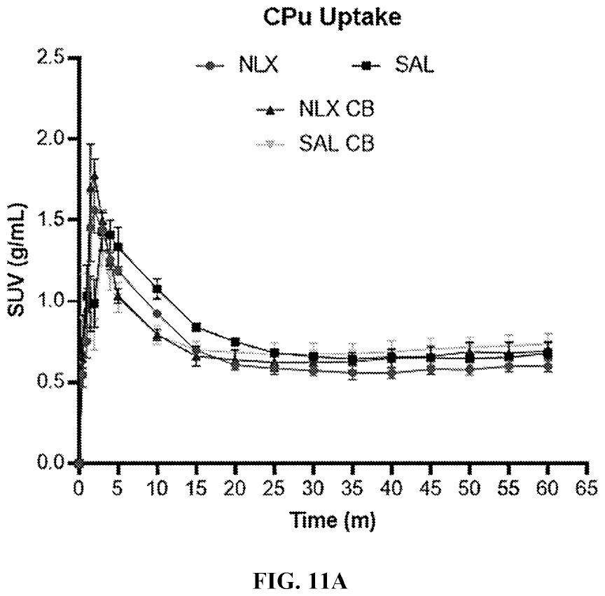

Production of [¹⁸F]fluoride. Oxygen-18 enriched water (98%, Huayi Isotopes, Jiangsu, China, approximately 2 mL) was loaded into a niobium body, high-yield [¹⁸F]fluoride target of a General Electric Medical Systems (GEMS, Uppsala, Sweden) PETtrace cyclotron. The target was irradiated with a proton beam of 60 μA for 30-37 minutes to produce and average of 55.4 GBq (1.5 Ci) (n=3) of aqueous [¹⁸F]fluoride ion.

[¹⁸F]fluoroethyl tosylate synthesis. The radiosynthesis was performed using a custom-made radiofluorination module (RFM) using LabView control software. Microwave heating was done using a CEM Corporation PETwave microwave (Matthews, NC). The cyclotron produced [¹⁸F]fluoride ion was collected in a 5 mL V-vial (Wheaton) inside the hot cell, and assayed in a dose calibrator to obtain the initial starting radioactivity. The SUBSTITUTE SHEET (RULE 26) [¹⁸F]fluoride ion was then remotely transferred to the RFM where it was trapped on a Chromafix 30-PS-HCO3 solid-phase extraction (SPE) cartridge (ABX GmbH, Radeberg, Germany) earlier preconditioned using 1 mL of high-purity water (Honeywell). The Water was collected for recycling. The resin cartridge was eluted using 150 μL of a 1:1 acetonitrile/water mixture containing 18.1 μmoles of potassium carbonate and 31.9 μmoles of Kryptofix K222 into an empty CEM 5-mL conical borosilicate glass reaction vessel. The resin cartridge was then rinsed with 250 microliters of acetonitrile into the same reaction vessel and the [¹⁸F]fluoride was dried at 110° C. with nitrogen flow (325 mL/min) for 150 seconds in a standard thermal heating block. Then two separate additions of 250 microliter acetonitrile were added with 150 second and 180 second drying, respectively. After drying, software environment (PMOD Technologies, Zurich Switzerland). The dynamic PET images were co-registered to MRI templates and time-activity curves were generated using PMOD's built-in atlases and the described analyses were performed. Standardized uptake value (SUV) was calculated as using the equation SUV=C/(dose/BW) where C is the tissue concentration of [¹⁸F]FNZ (kBq/cc), dose is the administered dose (MBq), and BW (kg) is the animal's body weight. Statistical analyses were performed using Prism 7 (GraphPad Software, La Jolla, CA, USA). Using the SUV, data were also expressed as Region/CB ratios. The results are set forth in FIGS. 10A, 10B, 11A, 11B, and 12.

FIGS. 10A and 10B show the lateral view (FIG. 10A) and dorsal view (FIG. 10B) of the accumulation of [¹⁸F]FNZ in the brain over 60 minutes of the rat treated with [¹⁸F]FNZ. As is apparent from the results set forth in FIGS. 10A and 10B, despite its high affinity, [¹⁸F]FNZ does not show any accumulation in the rat brain and only a small amount of non-specific accumulation in the harderian glands over a period of 60 minutes.

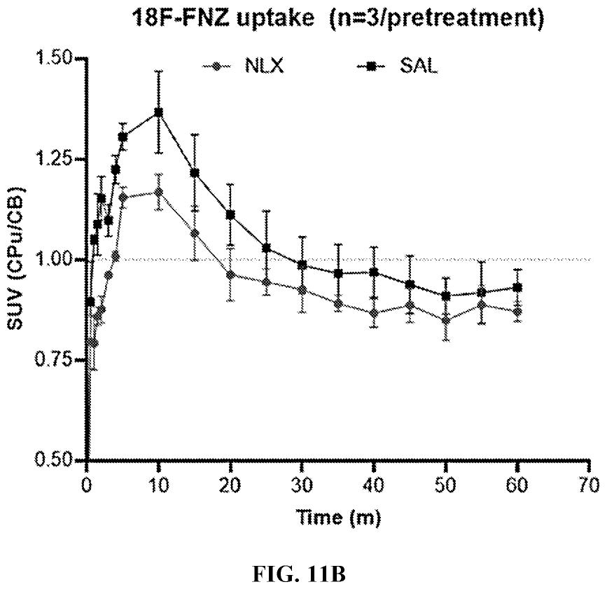

FIGS. 11A and 11B provide graphs showing the time activity curves of [¹⁸F]FNZ accumulation in the caudate putamen (CPu) (MOR-rich region) and cerebellum (CB) (MOR-poor region). FIG. 11A shows the CPu uptake and CB uptake of [¹⁸F]FNZ in rats and FIG. 11B shows the CPu/CB ratio of [¹⁸F]FNZ uptake in rats. As is apparent from the results set forth in FIG. 11A, and the CPu/CB ratios set forth in FIG. 11B, a small amount of [¹⁸F]FNZ is present in the CPu only in the first 10 min after its injection.

FIG. 12 shows the accumulation of [¹⁸F]FNZ specific binding (i.e., displaceable by naloxone) in the MOR-rich brain regions. As is apparent from the results set forth in FIG. 12, accumulation of [¹⁸F]FNZ specific binding (i.e., displaceable by naloxone) is observed in MOR-rich brain regions in the first 10 min after its injection confirming [¹⁸F]FNZ rapidly enters and exits the brain.

### Example 11

This example provides the brain uptake and distribution exhibited by rats treated with [¹⁸F]FNZ.

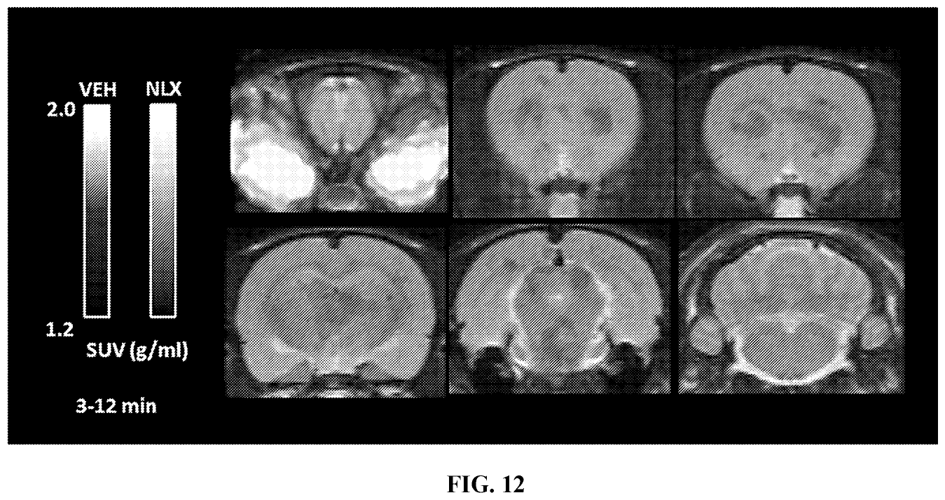

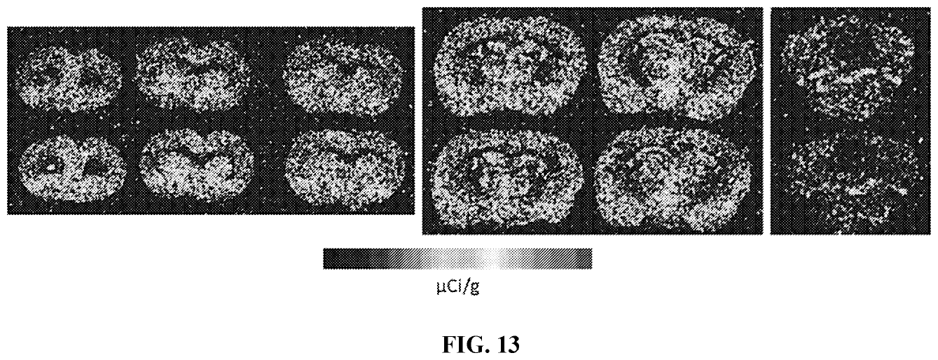

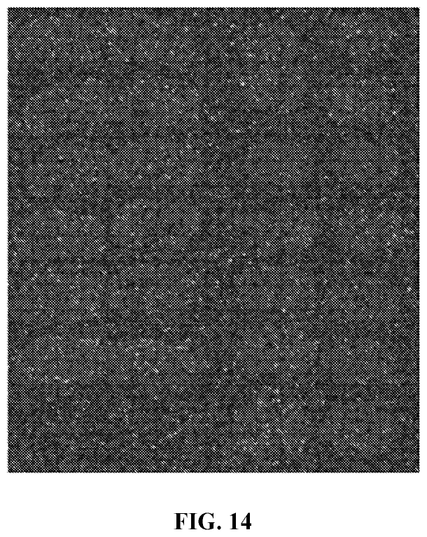

Male rats were injected (1 μg/kg administered intravenously (IV)) with [³H]FNZ and euthanized at 7 minutes post injection, and brain and blood were collected for radiometric analyses. Male mice were injected (10 μg/kg administered subcutaneously (SC)) and euthanized at 30 minutes post injection and brains were collected. The brains were flash frozen in 2-methylbutane and stored at −80° C. until use. The blood was centrifuged (13,000 rpm, 10 minutes at room temperature) and serum was collected. Serum samples were dissolved in scintillation cocktail (2.5 mL) and radioactivity counts were determined using a liquid scintillation counter. The brains were sectioned (20 μm) on a cryostat (Leica), mounted into glass microscope slides, and air-dried overnight at room temperature. The day after slides were placed into a Hypercassette™ covered by a BAS-TR₂₀₂₅ phosphor screen (FujiFilm; GE Healthcare). The slides were exposed to the phosphor screen for 15 days and imaged using a phosphor imager (Typhoon FLA 7000; GE Healthcare). The digitized images were calibrated using C-14 standard slides (American Radiolabeled Chemicals). ROIs were hand-drawn based on anatomical landmarks and radioactivity was quantified using ImageJ. The activity in 6-10 different sections was averaged per animal and brain region. The results for the treated rats are set forth in FIG. 13 and the results for the treated mice are set forth in FIG. 14.

As is apparent from the results set forth in FIG. 13, [³H]FNZ uptake is observed in mu-opioid receptor (MOR)-rich regions of the brain, but not in mu-opioid receptor (MOR)-poor regions of the brain such as the cerebellum. FIG. 14 shows that at approximately 30 minutes posit injection, no [³H]FNZ uptake was observed, indicating that [³H]FNZ was cleared from the brain in less than 30 minutes.

### Example 12

This example provides an assessment of the locomotor activity exhibited by mice treated with fluornitrazene (FNZ).

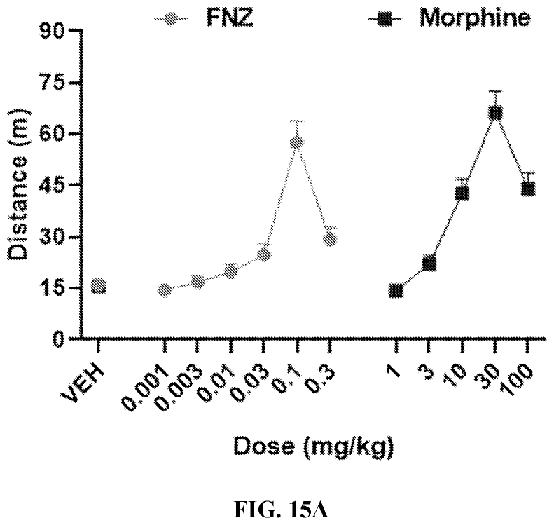

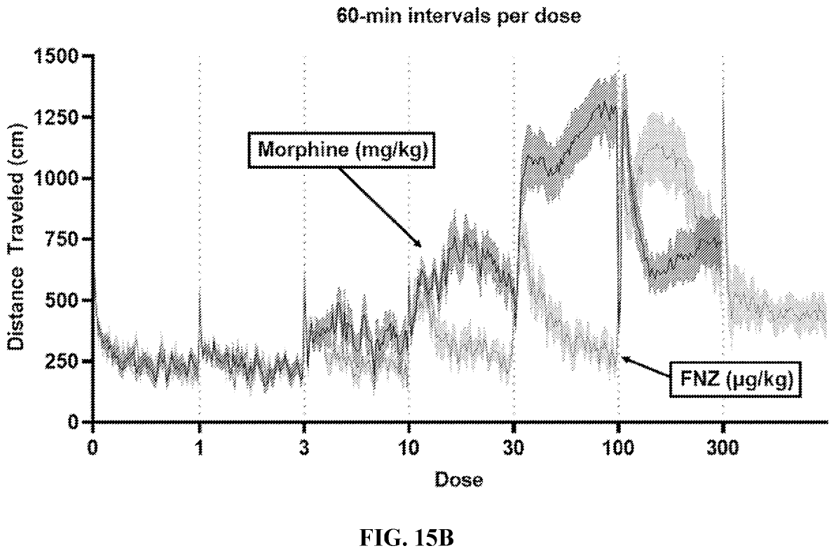

Locomotor activity was recorded immediately after the mice were introduced in the activity chambers in an open field arena (Opto-varimex ATM3, Columbus Instruments). After habituation and saline injections, they were systemically administered morphine (1 mg/kg, 3 mg/kg, 10 mg/kg, 30 mg/kg, or 300 mg/kg administered intraperitoneal (IP)) or FNZ (1 μg/kg, 3 μg/kg, 10 μg/kg, 30 μg/kg, 100 μg/kg, or 300 μg/kg administered intraperitoneal (IP)) and locomotor activity was recorded for an additional 60 minute period. The results are set forth in FIGS. 15A, which shows the cumulative distance traveled over each 60 minute session, and 15B, which shows the time-course of distance traveled during each 60 minute session.

As is apparent from the results set forth in FIGS. 15A and 15B, FNZ is more potent in inducing locomotion in mice compared to morphine. In particular, FNZ begins to produce locomotion at approximately 30 μg/kg, which is consistent with the occupancy and PET studies provided in Examples 10 and 11. In addition, FIG. 15B shows that the effects on locomotion exhibited by FNZ are short lived compared to morphine.

### Example 13

This example provides the results of a respiratory study, as measured using a pulse oximetry, exhibited by rats treated with fluornitrazene (FNZ).

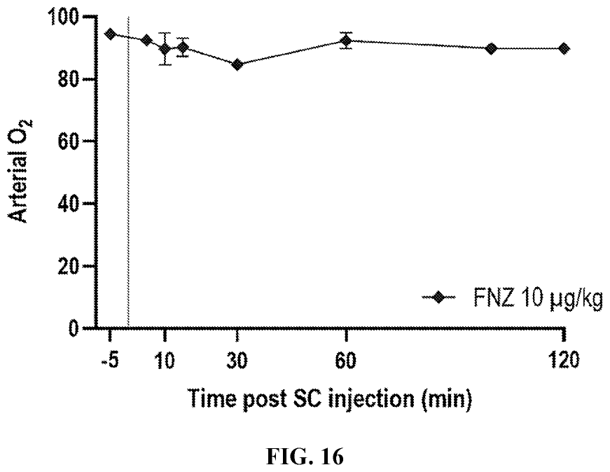

Oxygen saturation was measured in freely moving rats using a collar sensor and pulse oximetry (MouseOx Plus, Starr Life Sciences). Animals were habituated to experimenter handling and wearing oximeter collars daily for two weeks prior to testing. After baseline readings, two rats received FNZ (10 μg/kg administered subcutaneously (SC)), followed by repeated oximetry readings (0-240 minutes). Oximetry readings were averaged across a 60 second period for the respective time points. Experimenter was blinded to all drug conditions. The results are plotted in FIG. 16, wherein the vertical line denotes FNZ injection time.

As is apparent from the results set forth in FIG. 16, FNZ produces no apparent respiratory effects at a dose that produces maximum antinociception and is 2-fold greater than the hot plate ED50, as described in Example 15.

### Example 14

This example provides the results of two stable cell-based reporter assays used to assess the in vitro biological MOR activity of fluornitrazene (FNZ), etonitazene, fentanyl, hydromorphone, and DAMGO (i.e., a synthetic opioid peptide).

Activation of human MOR, fused to one part of a split nanoluciferase (NanoLuc Binary Technology, Promega), results in the recruitment of either β-arrestin2 (Barr2) (in the presence of coexpressed G protein-coupled receptor kinase 2, GRK2) or mini-Gi (GTPase domain of the Gai subunit), fused to the complementing part of the split nanoluciferase. The resulting functional complementation of the nanoluciferase restores its enzymatic activity, which, upon addition of the substrate furimazine, yields a measurable bioluminescent signal.

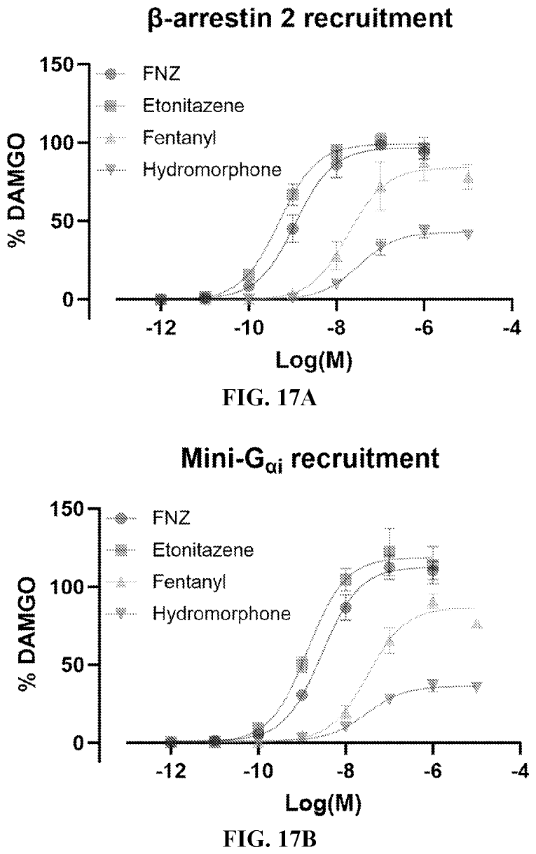

Cells expressing either MOR-Barr2-GRK2 (for simplicity, referred to as MOR-Barr2) or MOR-mini-Gi were seeded on poly-d-lysine coated 96-well plates (5×104 cells/well) 1 day prior to the experiments. Following overnight incubation, the cells were washed twice with Opti-MEM I reduced serum medium before adding 90 μL of OptiMEM. Nano-Glo Live Cell Reagent was then prepared by 20-fold dilution of Nano-Glo Live Cell Substrate with Nano-Glo LCS Dilution Buffer, and 25 μL was added to each well. The plate was subsequently placed into a multimode microplate reader and luminescence was continuously monitored until stabilization of the signal. Next, each test compound (i.e., FNZ, etonitazene, fentanyl, and hydromorphone) was added per well and luminescence was monitored. Compounds were tested in both assays, with appropriate solvent controls included in each experiment. Each compound was evaluated in three independent experiments (n=3), with duplicates or triplicates run for each concentration within an experiment to ensure the reliability of single values. The results for the β-arrestin2 recruitment assay and the mini-Gai recruitment assay are set forth in FIGS. 17A and 17B, respectively, and are summarized in Table 1.

TABLE 1
β-arrestin2 Recruitment Assay and Mini-Gαi Recruitment Assay Results

| | β-arrestin2 | | Mini-Gαi | |
|---|---|---|---|---|
| | EC₅₀ | Eₘₐₓ | EC₅₀ | Eₘₐₓ |
| | (nM) | (% DAMGO) | (nM) | (% DAMGO) |
| FNZ | 1.15 | 96.8 | 2.87 | 113 |
| | (0.875-1.52) | (93.0-101) | (2.22-3.71) | (109-117) |
| Etonitazene | 0.49 | 99.2 | 1.36 | 119 |
| | (0.383-0.624) | (96.1-102) | (0.946-1.96) | (113-125) |
| Fentanyl | 18.7 | 84.1 | 32.4 | 86.7 |
| | (11.0-32.6) | (77.4-91.1) | (21.7-48.0) | (81.2-92.4) |
| Hydromorphone | 31.3 | 43.0 | 30.2 | 36.4 |
| | (21.7-45.2) | (40.6-45.4) | (21.6-42.4) | (34.6-38.1) |

As is apparent from the results set forth in FIGS. 17A and 17B and Table 1, FNZ was more potent and produced greater efficacy than fentanyl and hydromorphone. In addition, although FNZ was slightly less potent than etonitazene, FNZ exhibited similar efficacy to etonitazene. However, both FNZ and etonitazene exhibited superagonism (i.e., higher efficacy that DAMGO, a synthetic opioid peptide) at MOR G protein signaling.

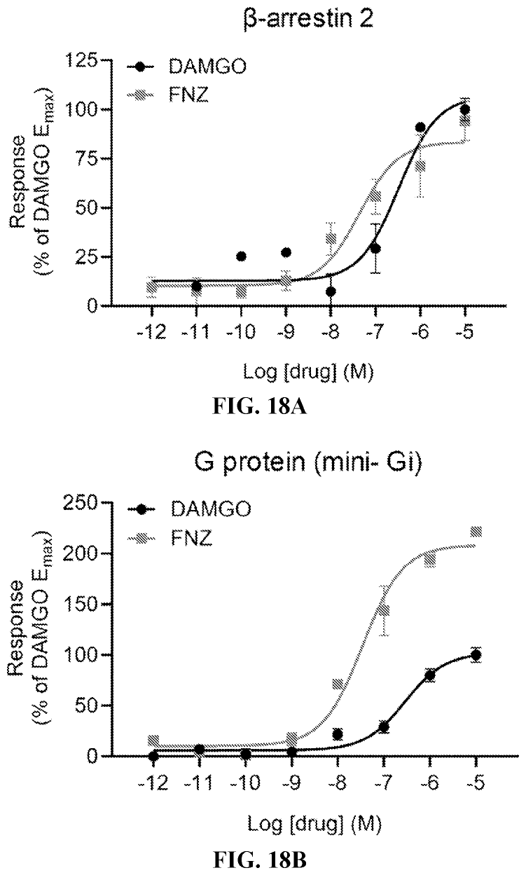

The foregoing experiment was repeated in a series of separate experiments using the same bioluminescent constructs transiently transfected into HEK293 cells. The results for the β-arrestin2 recruitment assay and the mini-Gai recruitment assay are set forth in FIGS. 18A and 18B, respectively, and are summarized in Table 2.

TABLE 2
β-arrestin2 Recruitment and Mini-Gαi Recruitment Results in HEK293 Cells

| | β-arrestin2 | | Mini-Gαi | |
|---|---|---|---|---|
| | EC₅₀ (nM) | Eₘₐₓ (% DAMGO) | EC₅₀ (nM) | Eₘₐₓ (% DAMGO) |
| FNZ | 43.3 | 94 | 34.6 | 222 |
| DAMGO | 328.1 | 100 | 288.8 | 100 |

As is apparent from the results set forth in FIGS. 18A and 18B and Table 2, FNZ was more potent but produced equivalent efficacy to DAMGO at MOR β-arrestin signaling. However, FNZ was more potent than DAMGO and produced superagonist efficacy at MOR G protein signaling. These results suggest that FNZ exhibits superagonism at MOR G protein signaling but not at MOR β-arrestin signaling. In this way, FNZ may be considered to exhibit “biased” superagonism of MOR G protein signaling.

### Example 15

This example provides the analgesia, catalepsy, and hypothermia effects exhibited by rats treated with fluornitrazene (FNZ).

On the day of an experiment, rats were brought into the laboratory in their home cages and allowed one hour to acclimate. Groups of rats (n=5 per dose group) received subcutaneous injections (SC) of vehicle (1 mL/kg saline) or FNZ (1 μg/kg, 3 μg/kg, 10 μg/kg, or 30 μg/kg) on the lower back between the hips and were returned to their home cages.

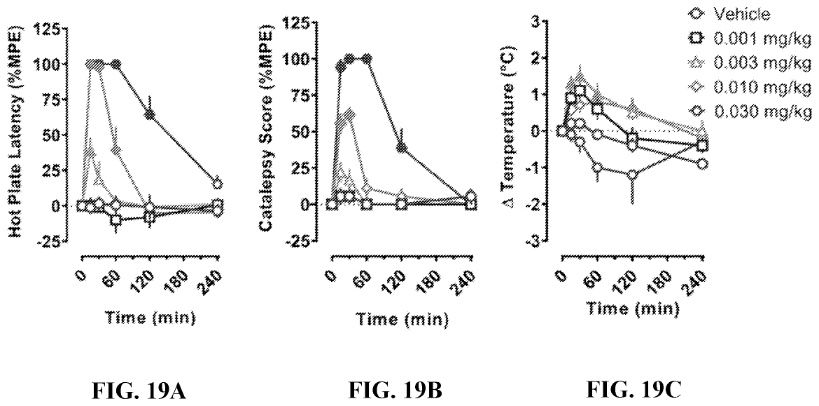

Each rat was tested twice in separate experimental sessions, with at least 3 days of washout between experiments, and doses were randomly assigned. Pharmacodynamic endpoints including catalepsy score, body temperature, and hot plate latency, were determined prior to injection and at 15, 30, 60, 120, and 240 minutes post-injection. At each time point, behavior was observed for 1 minute by an experienced rater, and catalepsy was scored based on three overt symptoms: immobility, flattened body posture, and splayed limbs. Each symptom was scored as either 1=absent or 2=present, and catalepsy scores at each time point were summed, yielding a minimum score of 3 and a maximum score of 6. Next, body temperature was measured using a hand-held reader sensitive to signals emitted by the surgically implanted transponder. Hypothermia was chosen as a representative adverse effect of opioid treatment, since body temperature is a physiological measure that decreases in parallel with opioid-induced bradycardia and respiratory depression. Finally, rats were placed on a hot plate analgesia meter (IITC Life Sciences, Woodland Hills, CA, U.S.) set at 52° C. Rats remained on the hot plate until they exhibited hind paw licking in response to the heat stimulus and were then returned to their home cages. Time spent on the hot plate was recorded using a timer triggered by a foot pedal. A 45 second cut-off was employed to prevent tissue damage. Pharmacodynamic findings were analyzed using GraphPad Prism 9. Raw time-course data for hot plate latency and catalepsy score were normalized to percent maximum possible effect (% MPE), using the following equation: (experimental measure-baseline measure)/(maximum possible response-baseline measure)×100. The maximum possible response for hot plate latency was 45 seconds, whereas the maximal response for catalepsy score was 6. Raw time-course data for body temperature were normalized to change from baseline, A temperature in ° C., for each rat. Normalized time-course data were analyzed by two-factor (dose×time) ANOVA followed by Tukey's post hoc test to determine effects of drug doses at each time point. Mean hot plate latency and catalepsy score over the first 60 minutes were used to construct dose-response relationships, which were analyzed by nonlinear regression (response stimulation, normalized response) to determine ED50 (potency) values. The analgesia, catalepsy, and hypothermia results are set forth in FIGS. 19A, 19B, and 19C, respectively.

As is apparent from the results set forth in FIGS. 19A-19C, FNZ produces maximum analgesic effects in mice on the hot plate at a 10 μg/kg subcutaneous dose, lasting at least 30 minutes with 50% efficacy at 60 minutes (ED50=5 μg/kg). At this dose, minimum catalepsy and no decrease in temperature is observed, which is consistent with its low occupancy levels and fast brain entry and clearance, as demonstrated by Examples 10-12. For comparison, Vandeputte et al. [Drug and Alcohol Dependence, 249, 109939(2023)] previously showed that fentanyl required 30 μg/kg subcutaneous dose to produce max analgesia in this same assay.

### Example 16

This example provides the results of a mechanical sensitivity (von Frey) test exhibited by rats treated with fluornitrazene (FNZ), as compared to rats treated with fentanyl.

Rats were handled and habituated to chambers for 15-30 minutes per day and received saline SC injections in the week prior to testing. Rats received daily received subcutaneous injections (SC) of vehicle (1 mL/kg saline), FNZ (3 μg/kg or 10 μg/kg), or fentanyl (10 μg/kg or 30 μg/kg) for two weeks (5 days per week). Since prior data indicated that FNZ is more potent than fentanyl, fentanyl was dosed ~3× higher than FNZ.

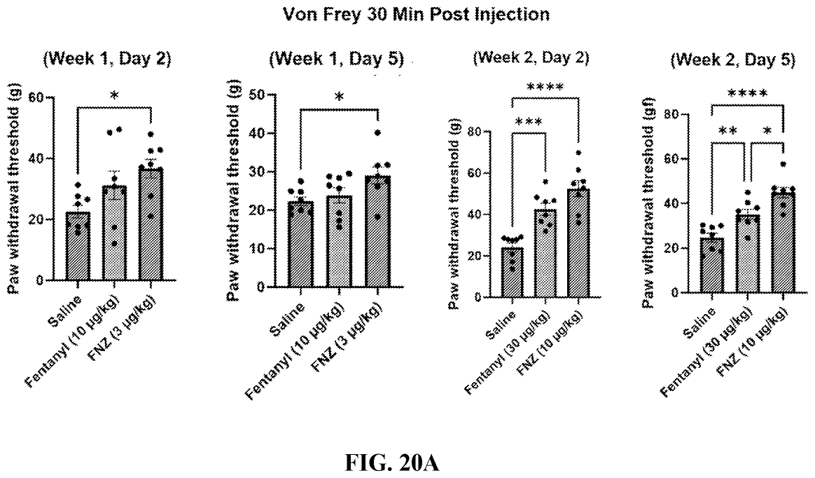

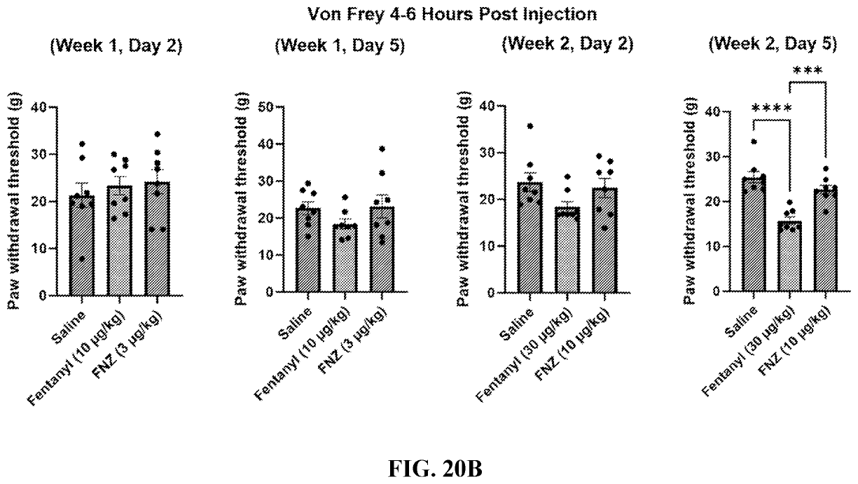

Rats were tested 2 times per week after drug administration (saline, FNZ, fentanyl) on days 2 and 5. Analgesia was tested 30 minutes post injection and hyperalgesia was tested 4-6 hours post injection. Mechanical sensitivity was measured twice per time point with at least 20 second intervals between acquisitions. Rats were habituated to the testing room for at least 30 minutes and acclimatized to the testing apparatus for at least 15 minutes before testing. The results of the 30 minute post-injection analgesia test and the 4-6 hour post-injection hyperalgesia test are set forth in FIGS. 20A and 20B, respectively. Data are shown as the mechanical threshold in (g) required for the rat to withdraw their stimulated paw. The dose-dependent effects of FNZ and fentanyl were analyzed by using one-way ANOVA with Tukey's multiple comparison tests.

As is apparent from the results set forth in FIG. 20A, at 30 minutes post-injection, FNZ significantly increased the paw withdrawal threshold (a measure of mechanical analgesia) in the von Frey procedure at 3 and 10 μg/kg. In contrast, fentanyl significantly increased the paw withdrawal threshold only at 30 μg/kg. The effect of 10 μg/kg FNZ was significantly greater than that of 30 μg/kg fentanyl.

As is apparent from the results set forth in FIG. 20B, at 4-6 hours post-injection, the dose of fentanyl that produced max analgesia in the hotplate (i.e., 30 μg/kg), and significant analgesia in von Frey at 30 minutes post-injection (FIG. 20A), also significantly decreased the paw withdrawal threshold indicating hyperalgesia. In contrast, the dose of 10 μg/kg FNZ, which produced maximum analgesia in the hotplate (FIG. 19A) and significantly higher analgesia in von Frey than 30 μg/kg fentanyl, as exhibited by FIG. 20A, did not produce hyperalgesia.

### Example 17

This example provides the effect of heroin self-administration and food self-administration exhibited by rats treated with fluornitrazene (FNZ), as compared to rats treated with fentanyl.

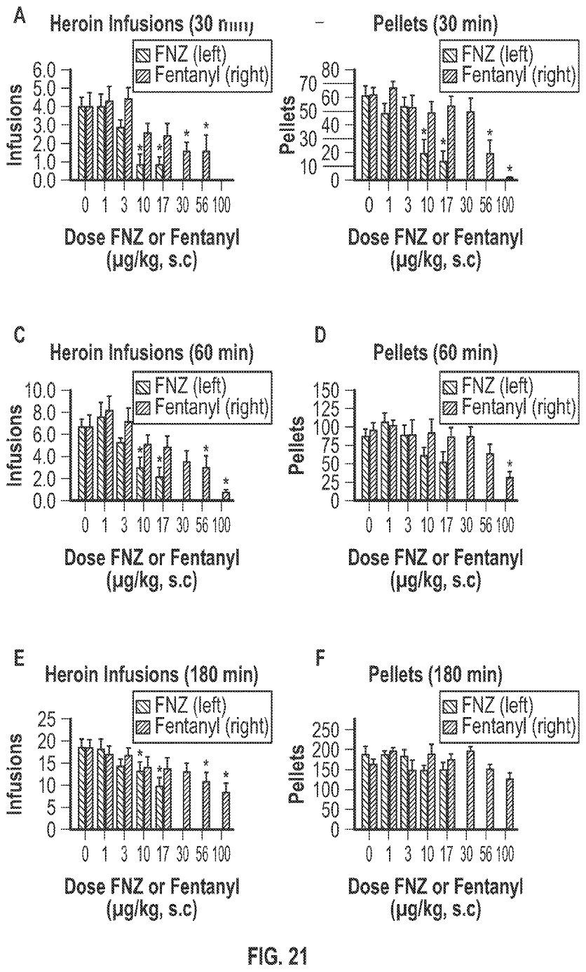

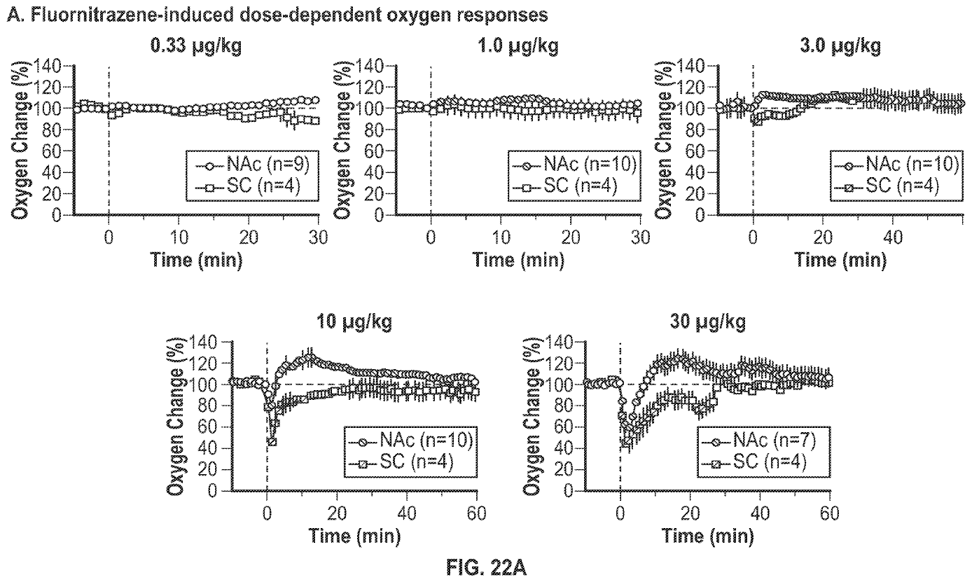

Rats were implanted with jugular vein catheters and trained to self-administer heroin or food. Subcutaneous injections of saline (1 mL/kg), FNZ (1 μg/kg, 3 μg/kg, 10 μg/kg, or 17 μg/kg), or fentanyl (1 μg/kg, 3 μg/kg, 10 μg/kg, 17 μg/kg, 30 μg/kg, 56 μg/kg, 100 μg/kg) were injected immediately prior to self-administration testing and behavioral responses were recorded for 30 minutes, 60 minutes, and 180 minutes. The results are set forth in FIG. 21A (heroin 30 minutes), 21B (food 30 minutes), 21C (heroin 60 minutes), 21D (food 60 minutes), 21E (heroin 180 minutes), and 21F (food 180 minutes). Data were analyzed using a mixed effects ANOVA.

As is apparent from the results set forth in FIGS. 21A-21F, both FNZ and fentanyl significantly decreased heroin (number of heroin infusions) and food (number of food pellets) self-administration assessed at 30 minutes after drug pretreatment, but FNZ was more potent than fentanyl in decreasing heroin and food self-administration. More particularly, FNZ was 3-5-fold more potent than fentanyl in decreasing heroin self-administration at 30 min. At 60 minutes after FNZ or fentanyl injection, FNZ significantly decreased heroin self-administration without significantly affecting food pellet self-administration. However, at 60 min administration of 100 μg/kg fentanyl significantly decreased food pellet self-administration. At 180 min after FNZ or fentanyl injection, both FNZ and fentanyl significantly decreased heroin self-administration but neither drug affected food pellet self-administration. These results show that FNZ may be able to serve as a substitute therapy for heroin dependency and opioid use disorders.

### Example 18

This example provides the results of a respiratory study exhibited by rats treated with fluornitrazene (FNZ), as compared to rats treated with fentanyl. The effects on the nucleus accumbens (NAc) space, which shows the oxygen levels in the brain, and the subcutaneous (SC) space, which shows the peripheral oxygen levels.

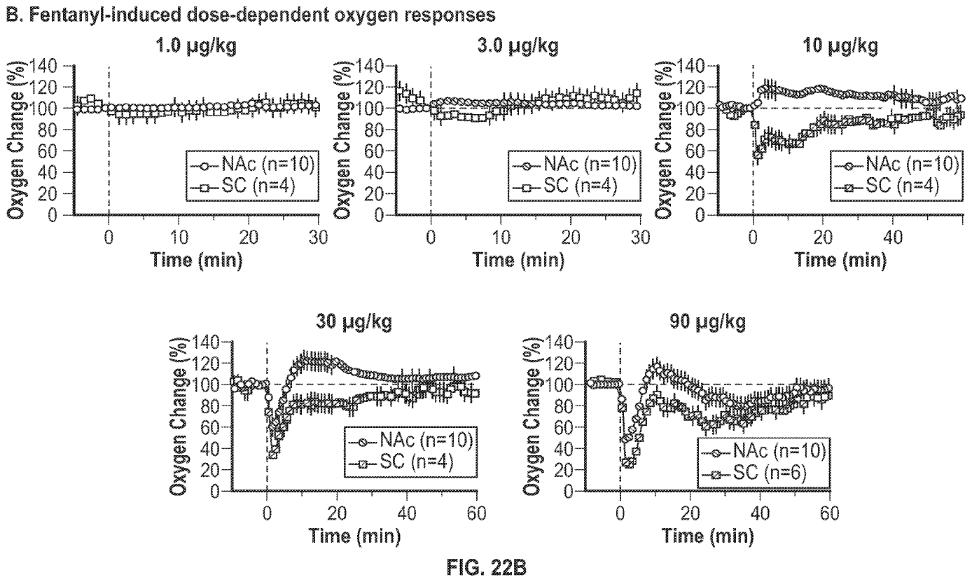

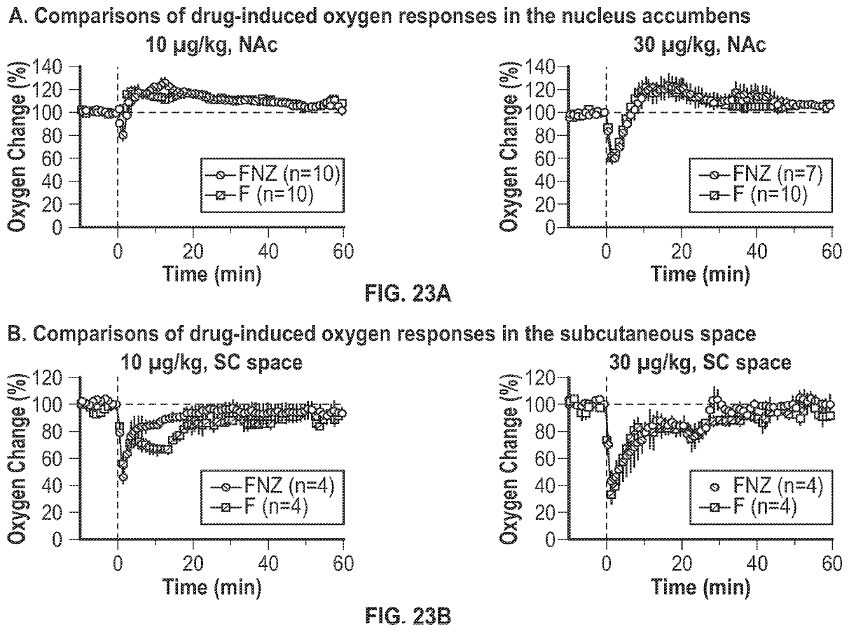

Rats were implanted with two oxygen sensors in the nucleus accumbens (NAc) and subcutaneous (SC) space and equipped with an intravenous catheter. Experiments began 4-5 days after the surgeries and continued over several daily sessions (i.e., about 3-5 days). Both FNZ (0.33 μg/kg, 1 μg/kg, 3 μg/kg, 10 μg/kg, or 30 μg/kg) and fentanyl (1 μg/kg, 3 μg/kg, 10 μg/kg, 30 μg/kg, or 90 μg/kg) were delivered via a slow, stress-free intravenous injection in ascending order (small-to-largest dose) via the catheter extension. The results are set forth in FIGS. 22A and 22B. Data are shown as changes in oxygen levels relative to pre-injection baseline (=100%). The dose-dependent effects of FNZ and fentanyl were analyzed by using one-way repeated measure ANOVA. Differences between the FNZ- and fentanyl-induced oxygen responses at the same dose (10 μg/kg or 30 μg/kg) were also analyzed using two-way repeated measure ANOVA. The results are set forth in FIGS. 23A and 23B. Sample sizes of the NAc responses are larger than those of the SC oxygen responses.

As is apparent from the results set forth in FIGS. 22A, 22B, 23A, and 23B, at equivalent doses, FNZ and fentanyl led to comparable decreases in brain oxygen, but fentanyl showed longer lasting decreases in peripheral oxygen compared to FNZ, particularly at doses of 10 μg/kg and 30 μg/kg.

Examples 14-18 show that FNZ was more potent and efficacious than fentanyl at activating the MOR, producing analgesia, and decreasing intravenous heroin self-administration. In contrast, FNZ was less potent than fentanyl in its capacity to produce decreases in oxygen (e.g., respiratory depression) in the brain and periphery. FNZ produced robust decreases in heroin self-administration but did not meaningfully affect food pellet self-administration at 60 and 180 min after its administration. At 3 μg/kg, FNZ produced significant analgesia without any effect on oxygen levels, food pellet self-administration, or hyperalgesia. Fentanyl produced significant analgesia at 30 μg/kg and at this dose (and even at the lower 10 μg/kg dose) led to significant and robust decreases in oxygen. Finally, fentanyl at the 30 μg/kg analgesic dose also produced hyperalgesia. In contrast, at 10 μg/kg, FNZ produced significantly greater analgesia than 30 μg/kg fentanyl but FNZ did not produce hyperalgesia.

Without wishing to be bound by any particular theory, it is believed that FNZ is the only known MOR agonist that does not seem to produce hyperalgesia at effective analgesic doses. The data indicates that FNZ shows a stronger analgesic profile and lower propensity to produce adverse effects than fentanyl. The selectivity of FNZ for the MOR, its rapid brain entry, fast onset of analgesic efficacy and low adverse effect profile, its rapid brain clearance and limited amount and duration of MOR occupancy, its lack of active metabolites, and its superagonism profile at MOR G protein signaling over β-arrestin signaling at the MOR collectively represent superior critical and unexpected attributes compared to existing clinically used MOR agonists such as fentanyl. Moreover, these attributes differ greatly from those of other etonitazene analogs which show strong and persistent respiratory effects and depression. See, for example, Malcom et al. [iScience, 26(7): 107121 (2023)]. The data further suggests that FNZ may have promise as a selective and potent MOR agonist with a wide therapeutic window and low propensity for adverse effects compared to existing MOR-based medicines.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A fluorinated compound of Formula (I): 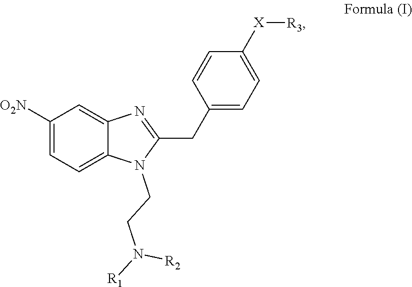
a deuterated or tritiated variant thereof, or a pharmaceutically acceptable salt thereof,
wherein X is optionally present and, when present, is —O—, —S—, or —NR₄—,
wherein each of R₁, R₂, R₃, and R₄ is independently selected from hydrogen, a substituted or unsubstituted C₁₋₆ alkyl group, a substituted or unsubstituted C₂₋₆ alkenyl group, a substituted or unsubstituted C₂₋₆ alkynyl group, a substituted or unsubstituted C₁₋₆ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, and a combination thereof, and
wherein at least one C—H bond on the compound of Formula (I) is replaced with a C—F bond or a C—¹⁸F bond.

2. The fluorinated compound of claim 1, a deuterated or tritiated variant thereof, or a pharmaceutically acceptable salt thereof, wherein X is —O—.

3. The fluorinated compound of claim 1, a deuterated or tritiated variant thereof, or a pharmaceutically acceptable salt thereof, wherein X is not present.

4. The fluorinated compound of claim 1, a deuterated or tritiated variant thereof, or a pharmaceutically acceptable salt thereof, wherein each of R₁, R₂, and R₃ is independently selected from C₁-C₆ alkyl, C₂-C₆ alkenyl, and C₂-C₆ alkynyl.

5. (canceled)

6. (canceled)

7. The fluorinated compound of claim 1, wherein the fluorinated compound is of Formula (Ia): 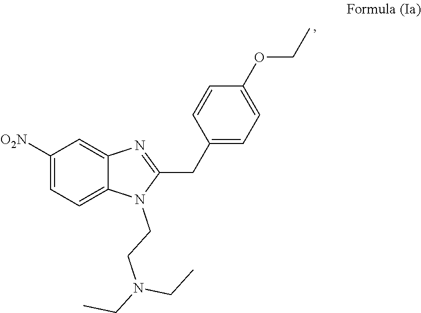 [SMILES: CCOc1ccc(Cc2nc3cc([N+](=O)[O-])ccc3n2CCN(CC)CC)cc1]
a deuterated or tritiated variant thereof, or a pharmaceutically acceptable salt thereof,
wherein at least one C—H bond on the compound of Formula (Ia) is replaced with a C—F bond or a C—¹⁸F bond.

8. The fluorinated compound of claim 1, wherein the fluorinated compound is of Formula (Ib): 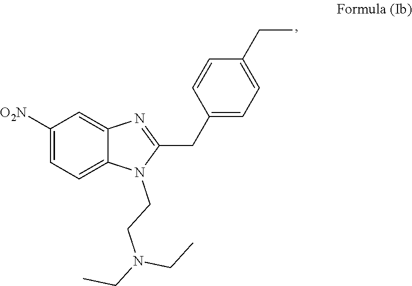 [SMILES: CCc1ccc(Cc2nc3cc([N+](=O)[O-])ccc3n2CCN(CC)CC)cc1]
a deuterated or tritiated variant thereof, or a pharmaceutically acceptable salt thereof,
wherein at least one C—H bond on the compound of Formula (Ib) is replaced with a C—F bond or a C—¹⁸F bond.

9. The fluorinated compound of claim 1, a deuterated or tritiated variant thereof, or a pharmaceutically acceptable salt thereof, wherein exactly one C—H bond on the compound of Formula (I) is replaced with a C—F bond or a C—¹⁸F bond.

10. (canceled)

11. The fluorinated compound of claim 1, a deuterated or tritiated variant thereof, or a pharmaceutically acceptable salt thereof, wherein the at least one C—H bond on the compound of Formula (I) that is replaced with a C—F bond or a C—¹⁸F bond is sp3 hybridized.

12. (canceled)

13. (canceled)

14. The fluorinated compound of claim 1, wherein the fluorinated compound is: 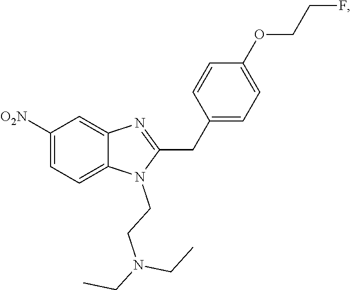 [SMILES: CCN(CC)CCn1c(Cc2ccc(OCCF)cc2)nc2cc([N+](=O)[O-])ccc21]
or a pharmaceutically acceptable salt thereof.

15. The fluorinated compound of claim 1, wherein the fluorinated compound is: 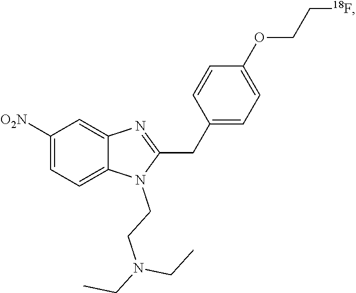
or a pharmaceutically acceptable salt thereof.

16. The fluorinated compound of claim 1, wherein the fluorinated compound is: 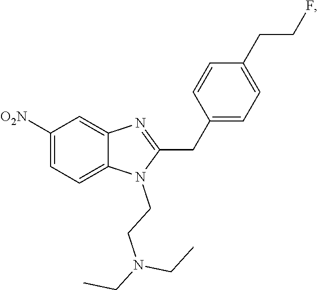 [SMILES: CCN(CC)CCn1c(Cc2ccc(CCF)cc2)nc2cc([N+](=O)[O-])ccc21]
or a pharmaceutically acceptable salt thereof.

17. The fluorinated compound of claim 1, wherein the fluorinated compound is: 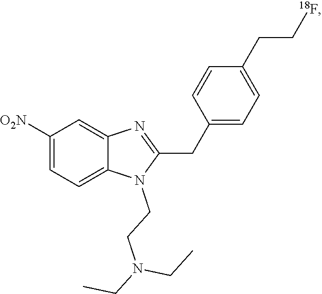
or a pharmaceutically acceptable salt thereof.

18. A pharmaceutical composition comprising a fluorinated compound of claim 1, a deuterated or tritiated variant thereof, or a pharmaceutically acceptable salt thereof, and a pharmaceutically acceptable carrier.

19-25. (canceled)

26. A method of treating or preventing pain in a subject, the method comprising administering to the subject a fluorinated compound of claim 1, a deuterated or tritiated variant thereof, or a pharmaceutically acceptable salt thereof, or a pharmaceutical composition comprising the fluorinated compound, a deuterated or tritiated variant thereof, or a pharmaceutically acceptable salt thereof, and a pharmaceutically acceptable carrier.

27. (canceled)

28. A method of treating opioid addiction or opioid use disorder in a subject, the method comprising administering to the subject a fluorinated compound of claim 1, a deuterated or tritiated variant thereof, or a pharmaceutically acceptable salt thereof, or a pharmaceutical composition comprising the fluorinated compound, a deuterated or tritiated variant thereof, or a pharmaceutically acceptable salt thereof, and a pharmaceutically acceptable carrier.

29. A method of monitoring mu-opioid receptor binding in a subject, the method comprising administering to the subject a fluorinated compound of claim 1, a deuterated or tritiated variant thereof, or a pharmaceutically acceptable salt thereof, or a pharmaceutical composition comprising the fluorinated compound, a deuterated or tritiated variant thereof, or a pharmaceutically acceptable salt thereof, and a pharmaceutically acceptable carrier, wherein at least one C—H bond on the fluorinated compound is replaced with a C—¹⁸F bond, and using positron emission tomography (PET) to determine mu-opioid receptor binding in the subject.

30. (canceled)

31. A method of anesthetizing a subject, the method comprising administering to the subject a fluorinated compound of claim 1, a deuterated or tritiated variant thereof, or a pharmaceutically acceptable salt thereof, or a pharmaceutical composition comprising the fluorinated compound, a deuterated or tritiated variant thereof, or a pharmaceutically acceptable salt thereof, and a pharmaceutically acceptable carrier.

32-36. (canceled)

37. The fluorinated compound of claim 1, wherein the fluorinated compound is: 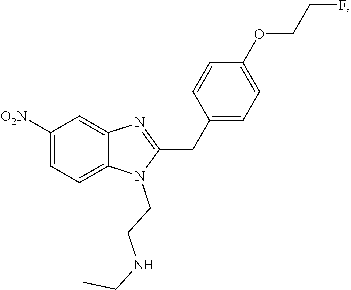 [SMILES: CCNCCn1c(Cc2ccc(OCCF)cc2)nc2cc([N+](=O)[O-])ccc21]
or a pharmaceutically acceptable salt thereof.

38. The fluorinated compound of claim 1, wherein the fluorinated compound is: 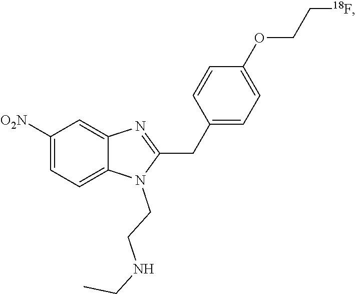
or a pharmaceutically acceptable salt thereof.

39. A compound of formula 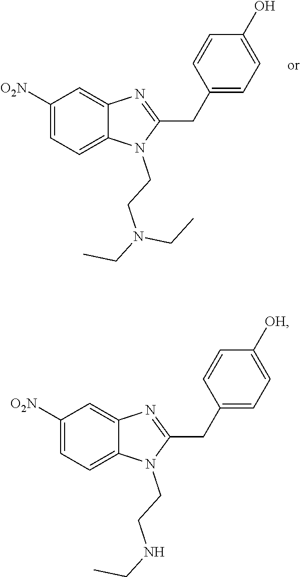 [SMILES: CCN(CC)CCn1c(Cc2ccc(O)cc2)nc2cc([N+](=O)[O-])ccc21 ; CCNCCn1c(Cc2ccc(O)cc2)nc2cc([N+](=O)[O-])ccc21]
or a pharmaceutically acceptable salt thereof.
